(19) United States
(12) Patent Application Publication
(10) Pub. No.: US 2002/0042140 A1
(43) Pub. Date: Apr. 11, 2002

(54) Methods for analysis of heterogeneous catalysts in a multi-variable screening reactor

(76) Inventors: Alfred Hagemeyer, Sunnyvale, CA (US); Anthony F. Volpe JR., Santa Clara, CA (US); Claus Lugmair, San Jose, CA (US); David M. Lowe, Mt. View, CA (US); Yumin Liu, San Jose, CA (US); H. Sam Bergh, San Francisco, CA (US); Shenheng Guan, Palo Alto, CA (US); Daniel M. Pinkas, Alameda, CA (US); Kyle W. Self, San Jose, CA (US); James R. Engstrom, Ithaca, NY (US); Laurent Lefort, Ashland, OR (US)

Correspondence Address: SYMYX TECHNOLOGIES INC, LEGAL DEPARTMENT, 3100 CENTRAL EXPRESS, SANTA CLARA, CA, 95051

(21) Appl. No.: 09/901,858

(22) Filed: Jul. 9, 2001

Related U.S. Application Data
(63) Continuation-in-part of application No. 09/518,794, filed on Mar. 3, 2000.
(63) Continuation-in-part of application No. 09/801,390, filed on Mar. 7, 2001.
(63) Continuation-in-part of application No. 09/518,794, filed on Mar. 3, 2000.
(63) Continuation-in-part of application No. 09/801,389, filed on Mar. 8, 2001.
(60) Provisional application No. 60/216,777, filed on Jul. 7, 2000.
(60) Provisional application No. 60/122,704, filed on Mar. 3, 1999.
(60) Provisional application No. 60/187,566, filed on Mar. 7, 2000.
(60) Provisional application No. 60/229,984, filed on Sep. 2, 2000.
(60) Provisional application No. 60/187,566, filed on Mar. 7, 2000.
(60) Provisional application No. 60/229,984, filed on Sep. 2, 2000.

(51) Int. Cl. G01N 33/00

(52) U.S. Cl. 436/34

(57) ABSTRACT

Protocols for designing and implementing sets of simultaneous experiments, in a parallel, multi-variable process optimization reactor, are disclosed. The multi-variable process optimization reactor is preferably a parallel flow reactor having the operational capability to simultaneously vary reaction conditions between reaction vessels—either modularly or independently. The simultaneously varied reaction conditions preferably include at least two of the following, in various combinations and permutations: space velocity, contact time, temperature, pressure and feed composition. Compositional variations in the catalysts residing in each of the reaction vessels can also be investigated in the set of simultaneous experiments implemented in the parallel reactor. Sufficient data is obtained from a single set of simultaneous experiments to generate a master curve.

This application claims the benefit of co-owned, co-pending U.S. provisional patent application Ser. No. 60/216,777 entitled “High-Throughput Methods for Evaluating Heterogeneous Catalysts” filed Jul. 7, 2000 by Hagemeyer et al., which is hereby incorporated by reference in its entirety for all purposes, and additionally, this application is a continuation-in-part of, and claims priority to, each of the following U.S. co-owned, co-pending, U.S. patent applications: (i) Ser. No. 09/518,794, entitled “Chemical Processing Microsystems, Diffusion-Mixed Microreactors and Methods for Preparing and Using Same”, filed Mar. 3, 2000 by Bergh et al. (which itself claims priority to commonly owned, co-pending U.S. patent application Ser. No. 60/122,704 filed Mar. 3, 1999 entitled “Chemical Processing Microsystems, Diffusion-Mixed Microreactors and Methods for Preparing and Using Same”); (ii) Ser. No. 09/801,390 entitled “Parallel Flow Process Optimization Reactor” filed Mar. 7, 2001 by Bergh et al (which itself is a continuation-in-part application of the aforementioned U.S. Ser. No. 09/518,794, and claims priority to co-owned U.S. patent application Ser. No. 60/187,566 entitled “Apparatus and Methods for Multi-Variable Optimization of Reaction Systems and Other Chemical Processing Microsystems”, filed Mar. 7, 2000 by Bergh et al., and to co-owned U.S. patent application Ser. No. 60/229,984 entitled “Apparatus and Methods for Optimization of Process Variables in Reaction Systems and Other Chemical Processing Systems”, filed Sep. 2, 2001 by Bergh et al.); and Ser. No. 09/801,389 entitled “Parallel Flow Reactor Having Variable Feed Composition” filed Mar. 7, 2001 by Bergh et al.(which itself claims priority to the aforementioned co-owned U.S. patent applications Ser. No. 60/187,566 and Ser. No. 60/229,984), each of which is hereby incorporated by reference in its entirety for all purposes.

### BACKGROUND OF THE INVENTION

Heterogenous catalysts have a variety of known applications, in diverse fields including commodity chemicals and fine chemicals. It has long been recognized, however, that the catalytic activity and/or selectivity of heterogeneous catalysts can vary substantially due to many factors. Factors known to have a potential effect on catalytic activity and/or selectivity are described, for example, by Wijngaarden et al., “Industrial Catalysts—Optimizing Catalysts and Processes”, Wiley-VCH, Germany (1998).

Combinatorial (i.e., high-throughput) approaches for evaluation of catalysts and/or process conditions are also known in the art. See, for example, U.S. Pat. No. 5,985,356 to Schultz et al, U.S. Pat. No. 6,004,617 to Schultz et al., U.S. Pat. No. 6,030,917 to Weinberg et al., U.S. Pat. No. 5,959,297 to Weinberg et al., U.S. Pat. No. 6,149,882 to Guan et al., U.S. Pat. No. 6,087,181 to Cong, U.S. Pat. No. 6,063,633 to Willson, U.S. Pat. No. 6,175,409 to Nielsen et al., and PCT patent applications WO 00/09255, WO 00/17413, WO 00/51720, WO 00/14529, each of which U.S. patents and each of which PCT patent applications, together with its corresponding U.S. application(s), is hereby incorporated by reference in its entirety for all purposes. Considered individually and cumulatively, these references teach the synthesis and screening of arrays of diverse materials, and generally, of spatially-determinative arrays of diverse materials. Typical approaches involve primary synthesis and screening (high-throughput “discovery” screening) followed by secondary synthesis and screening (more moderate-throughput “optimization” screening), and optionally, followed by ternary synthesis and screening (e.g., typically traditional “bench scale” screening). These references also describe screening strategies in which compositionally-varying arrays are prepared (e.g., as part of a primary or secondary screen) first with broadly-varied gradients. Subsequently, “focused” libraries comprising more narrowly-varied gradients are prepared and screened (e.g., at the same level of screen) based on the results of the first screen. Such libraries or arrays of diverse materials such as catalysts can comprise binary, ternary and higher order compositional variations. See, for example, WO 00/17413 (as well as its corresponding U.S. application, Ser. No. 09/156,827 filed Sep. 18, 1998 by Giaquinta et al.) and WO 00/51720, (as well as its corresponding U.S. application, Ser. No. 09/518,794 filed Mar. 3, 2000 by Bergh et al.), each of which U.S. and PCT applications are hereby incorporated by reference in its entirety for all purposes.

More recently, high-throughput process optimization, including process optimization in parallel flow reactors has also been described. See, for example, WO 00/51720, (as well as its corresponding U.S. application, Ser. No. 09/518,794 filed Mar. 3, 2000 by Bergh et al.). Additional recent developments include parallel flow reactors suitable for evaluating heterogeneous catalysts and having substantial capability for simultaneous, multi-variable optimization. See, for example, U.S. patent applications Ser. No. 60/185,566 filed Mar. 7, 2000 by Bergh et al., Ser. No. 60/229,984 filed Sep. 2, 2000 by Bergh et al., Ser. No. 09/801,390 filed Mar. 7, 2001 by Bergh et al., and Ser. No. 09/801,389 filed Mar. 7, 2001 by Bergh et al., each of which U.S. and PCT applications are hereby incorporated by reference in its entirety for all purposes.

The efficiency of a catalyst discovery program is, in general, limited by rate-limiting steps of the overall process work flow. Additionally, high throughput approaches still require substantial efforts to explore vast compositional space. As such, current approaches, while offering substantial advances over previous traditional, lower-throughput approaches, can still be improved with respect to overall efficiency. Hence, there is a need in the art for improved overall research work flows for developing and evaluating heterogeneous catalysts for a particular reaction of interest. In particular, a need exists for more efficient, meaningful approaches for identifying new heterogeneous catalysts.

More specifically, a need exists for improved protocols for efficiently evaluating heterogeneous catalysts in multi-variable process optimization reactors. Although substantial advances have been made with respect to reaction-based screening of such catalyst candidates, relatively fewer advances have focused on how to apply such evolving screening capabilities.

### SUMMARY OF INVENTION

It is therefore an object of the present invention to provide improved methodologies for evaluating catalysts, and especially heterogeneous catalysts, in multi-variable process optimization reactors. It is also an object of the invention to provide for reaction-based screening strategies and protocols that improve the overall efficiency of a combinatorial (i.e., high-throughput) catalysis research program, especially a heterogeneous catalysis research program.

Briefly, therefore, the present invention is directed to methods for evaluating process conditions for a catalyzed chemical reaction in a parallel flow reactor. The parallel flow reactor comprises four or more reaction vessels, and preferably six or more reaction vessels. One or more reactants are simultaneously supplied through a fluid distribution system to each of four or more, preferably six or more reactors of the parallel flow reactor under reaction conditions to effect a chemical reaction of interest. The reaction conditions are controllably varied between each of the four or more, preferably six or more reactors, such that meaningful master-curve (selectivity vs. conversion) data can be obtained in a single set of simultaneous experiments. The controllably variable set of reaction conditions associated with each reaction zone can preferably include two or more of space velocity, contact time, temperature, pressure and feed composition. A reactor effluent containing one or more reaction products and, in some cases, one or more unreacted reactants is simultaneously discharged from each of the six or more reactors. Each of the four or more, preferably six or more reactor effluent streams can be sampled and analyzed, preferably simultaneously analyzed (of each flow channel or of some subset thereof), to determine the conversion of one or more of the reactants, and the selectivity for at least one reaction product for the reaction of interest. Each of the six or more reactors comprises a catalyst having activity for the chemical reaction of interest. In general, depending on the number of reaction vessels in the parallel reactor, at least two or more, preferably at least four or more, and in some embodiments at least six or more of the catalysts are substantially the same—such that they have substantially the same composition and/or were prepared by substantially the same synthesis protocols with substantially the same compositional recipe and/or were prepared by substantially the same mechanical (e.g., grinding, pressing, crushing, sieving) treatments, chemical treatments, and/or physical treatments.

In preferred embodiments, the one or more sets of reaction conditions are controllably varied such that a determined conversion (e.g., the conversion of the conversion-limiting reactant) and a determined selectivity for one or more reaction products of the reaction of interest includes at least two, preferably at least three, preferably at least four, more preferably at least five and most preferably at least six data values for each of the catalyst compositions being evaluated. The determined data values for conversion (e.g., of a conversion-limiting reactant) preferably span a range of values that relate to the conversion range of interest for the chemical reaction, such that a meaningful master curve can be generated for each of the catalyst compositions being evaluated. Generally, the determined conversion values include four or more values, preferably six or more values, that span a range of at least about 5% conversion, preferably at least about 10% conversion difference, and more preferably at least about 20% conversion difference between the highest and lowest of such values, and for many reactions of interest, even more preferably at least about 30%, 40%, 50% or 60% conversion difference between the highest and lowest of such values. In some embodiments, the determined conversion values (e.g., of the conversion-limiting reactant) include four or more values, preferably six or more values ranging from less than about 20% conversion to more than about 40% conversion. Preferably, the set of reaction conditions are varied in the single set of simultaneous parallel reactions such that the determined conversion values (e.g., of the conversion-limiting reactant) include a range of four or more values, preferably six or more values, ranging from less than about 15% conversion to more than about 45% conversion, preferably ranging from less than about 10% conversion to more than about 50% conversion, and in some embodiments, ranging from less than about 10% conversion to more than about 70% conversion, or even ranging from less than about 10 % conversion to more than about 80% conversion. For some reactions of interest, relatively lower conversions are commercially significant, and therefore, the determined conversion values (e.g., of the conversion-limiting reactant) include three or more values, preferably four or more values, more preferably six or more values ranging from about 2% or less to more than about 5%, preferably from about 1% or less to more than about 10% conversion, more preferably from about 1% or less to more than about 15%.

According to one preferred method, a parallel reactor having six or more reaction vessels is loaded with a set of six or more catalyst having substantially the same composition. A set of reaction conditions is controllably varied between the six or more reaction vessels. In a variation of this embodiment, a parallel reactor having twelve or more reaction vessels is loaded with a set of twelve or more catalysts having substantially the same composition. A first set of reaction conditions is controllably varied between a first set of six or more reactors, and a second set of reaction conditions is controllably varied between a second set of six or more reactors. In each of these variations, the particular combinations of varied reaction conditions can include permutations of the binary and tertiary and quaternary combinations of space velocity, contact time, temperature, pressure and feed composition. Variations in feed composition with one or more of the other parameters are particularly preferred.

In another preferred embodiment, a parallel reactor having twelve or more reaction vessels is loaded with twelve or more catalysts—each having activity for the chemical reaction of interest. A first set of the twelve or more catalysts can be six or more first catalysts that have substantially the same first composition, and a second set of the twelve or more catalysts can be six or more second catalysts that have substantially the same second composition. A first set of reaction conditions is controllably varied between the reaction vessels comprising the first set of catalysts, and simultaneously therewith, a second set of reaction conditions is controllably varied between the reaction vessels comprising the second set of catalysts. For each of the first and second sets of reaction conditions, the particular combinations of varied reaction conditions can include permutations of the binary and tertiary and quaternary combinations of space velocity, contact time, temperature, pressure and feed composition. Variations in feed composition with one or more of the other parameters are particularly preferred.

In a further preferred embodiment, a parallel reactor having sixteen or more reaction vessels is loaded with sixteen or more catalysts—each having activity for the chemical reaction of interest. The sixteen or more catalysts include at least four sets of catalysts, each set having four or more catalysts, including a first set of four or more of the catalysts having substantially the same first composition, a second set of four or more of the catalysts having substantially the same second composition, a third set of four or more of the catalysts having substantially the same third composition, and a fourth set of four or more of the catalysts having substantially the same fourth composition. A set of reaction conditions is controllably varied between the sixteen or more reaction vessels—preferably such that at least one catalyst from each of the first, second, third and fourth sets of catalysts sees each of the varied reaction condition parameters, although not necessarily each combination of reaction conditions. In a preferred variation on this embodiment, the parallel reactor has twenty-four or more reaction vessels. Twenty-four or more catalysts, each having activity for the reaction of interest, are employed as four sets of catalysts, with each set having six or more catalysts of substantially the same composition. The particular combinations of varied reaction conditions can include permutations of the binary and tertiary and quaternary combinations of space velocity, contact time, temperature, pressure and feed composition. Variations in feed composition with one or more of the other parameters are particularly preferred.

For each of the aforedescribed preferred embodiments, exemplary binary combinations include, independently: (i) simultaneous controlled variations in contact time and one of temperature, pressure, space velocity or feed composition; (ii) simultaneous controlled variation in space velocity and one of temperature, pressure, contact time, or feed composition; (iii) simultaneous controlled variations in feed composition and one of space velocity, contact time, temperature or pressure; and (iv) simultaneous controlled variations in temperature and one of pressure, space velocity, contact time and feed composition. Exemplary tertiary combinations of reaction parameters include, independently: (i) simultaneous controlled variations in contact time, and two of temperature, pressure, space velocity or feed composition; (ii) simultaneous controlled variation in space velocity and two of temperature, pressure, contact time, or feed composition; (iii) simultaneous controlled variations in feed composition and two of space velocity, contact time, temperature or pressure; and (iv) simultaneous controlled variations in temperature and two of pressure, space velocity, contact time and feed composition, except in each case, that only two of space velocity, contact time and feed composition are independently varied. Exemplary quaternary combinations of reaction parameters include, independently: (i) simultaneous controlled variations in contact time, and three of temperature, pressure, space velocity or feed composition; (ii) simultaneous controlled variation in space velocity and three of temperature, pressure, contact time, or feed composition; (iii) simultaneous controlled variations in feed composition and three of space velocity, contact time, temperature or pressure; and (iv) simultaneous controlled variations in temperature and three of pressure, space velocity, contact time and feed composition, except, in each case, that only two of space velocity, contact time and feed composition are independently varied.

The present invention offers substantial advantages over many aspects of current combinatorial (i.e., high-throughput) research approaches. In particular, it allows for very rapid, single-experiment evaluation of catalysts in a complex, multiple-variable environment.

Although described herein primarily in the context of heterogeneous catalysis research, more specifically, in connection with heterogeneous catalysis evaluation in parallel flow reactors, and more particularly in heterogeneous catalysis evaluation of gas-phase chemical reactions, it will be appreciated that many aspects of the inventions disclosed and claimed herein also find application in connection with homogeneous catalysis research, with heterogeneous or homogeneous catalysis evaluation in semi-continuous or batch reactors, and in heterogeneous or homogeneous catalysis evaluation of liquid-phase chemical reactions or in two-phase or three-phase chemical reactions such as gas-liquid chemical reactions (e.g., in a plurality of trickle-bed reactors).

Other features, objects and advantages of the present invention will be in part apparent to those skilled in art and in part pointed out hereinafter. All references cited in the instant specification are incorporated by reference for all purposes. Moreover, as the patent and non-patent literature relating to the subject matter disclosed and/or claimed herein is substantial, many relevant references are available to a skilled artisan that will provide further instruction with respect to such subject matter.

### BRIEF DESCRIPTION OF THE DRAWINGS

FIG. 1 is a schematic diagram indicating the major steps in a comprehensive combinatorial (i.e., high-throughput) research program for heterogeneous catalysis.

FIG. 2A through FIG. 2T include schematic diagrams representing a parallel, multi-variable process optimization reactor (FIG. 2A), several schematic representations of experimental protocols for a simultaneous set of experiments in a parallel multi-variable process optimization reactor (FIG. 2B through FIG. 2E), and several schematic representations, in various detail, of a parallel, multi-variable process optimization reactor (FIG. 2F through FIG. 2T).

FIGS. 3A and 3B are schematic diagrams representing catalyst evaluation protocols that include prescreening of a representative composition at various process conditions (FIG. 3A), with subsequent evaluation of a related compositional library at a determined preferred process condition (FIG. 3B).

FIGS. 4A and 4B are schematic diagrams representing catalyst evaluation protocols that include preparing a representative composition under various synthesis conditions (FIG. 4A), with subsequent evaluation of the variously-prepared representative composition at various process conditions (FIG. 4B).

FIGS. 5A through 5E are a schematic representation of an combinatorial experiment design involving varied process conditions and a library comprising varied compositions including repeated samples of a representative composition (FIG. 5A) and plots showing a master curve of determined selectivity values versus determined conversion values for simultaneous evaluation of the varied process conditions and varied compositions in the library with predictive interpolation or extrapolation (FIG. 5B through FIG. 5E).

FIG. 6 is a plot showing master curves of determined selectivity values versus determined conversion values for each of three catalyst compositions (c1, c2, c3) evaluated at systematically varied process conditions.

FIGS. 7A and 7B are schematic diagrams representing catalyst library synthesis schemes, including prior art single-pot synthesis protocols (FIG. 7A) and selective, active-phase synthesis protocols (FIG. 7B).

The invention is described in further detail below with reference to the figures, in which like items are numbered the same in the several figures.

### DETAILED DESCRIPTION OF THE INVENTION

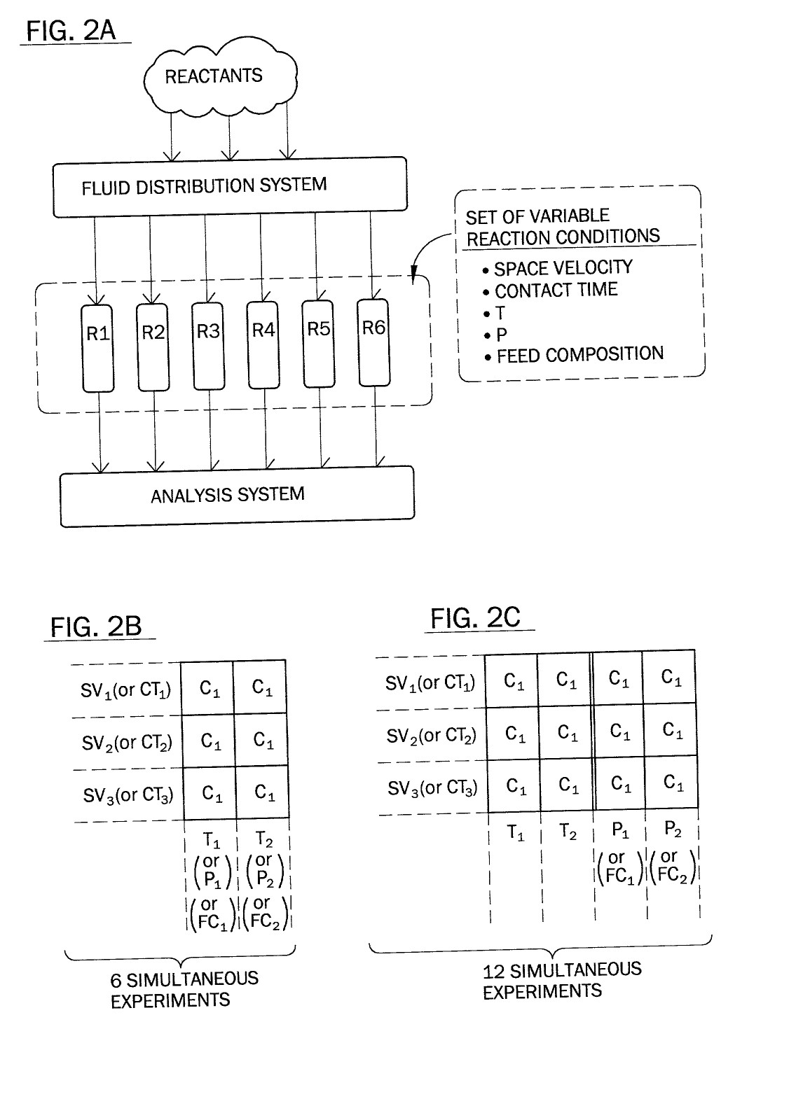

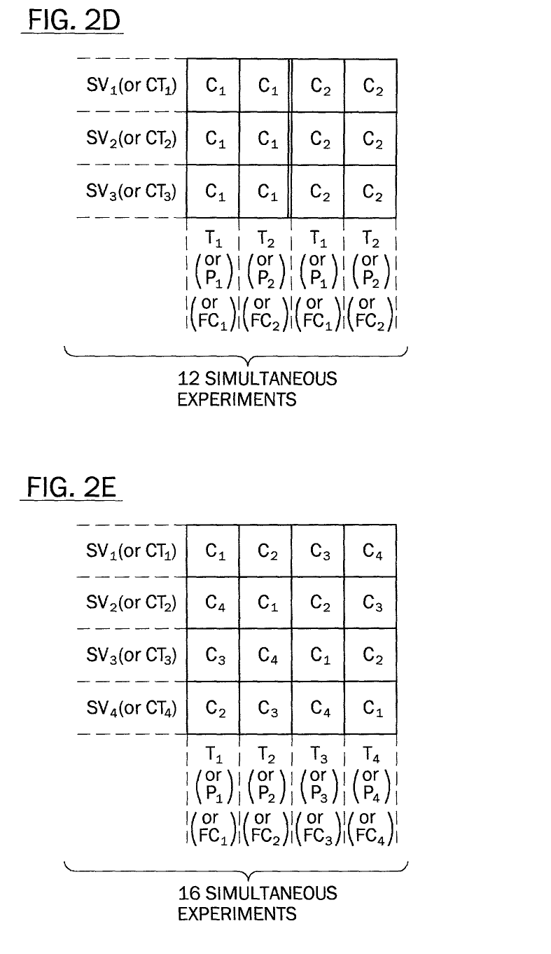

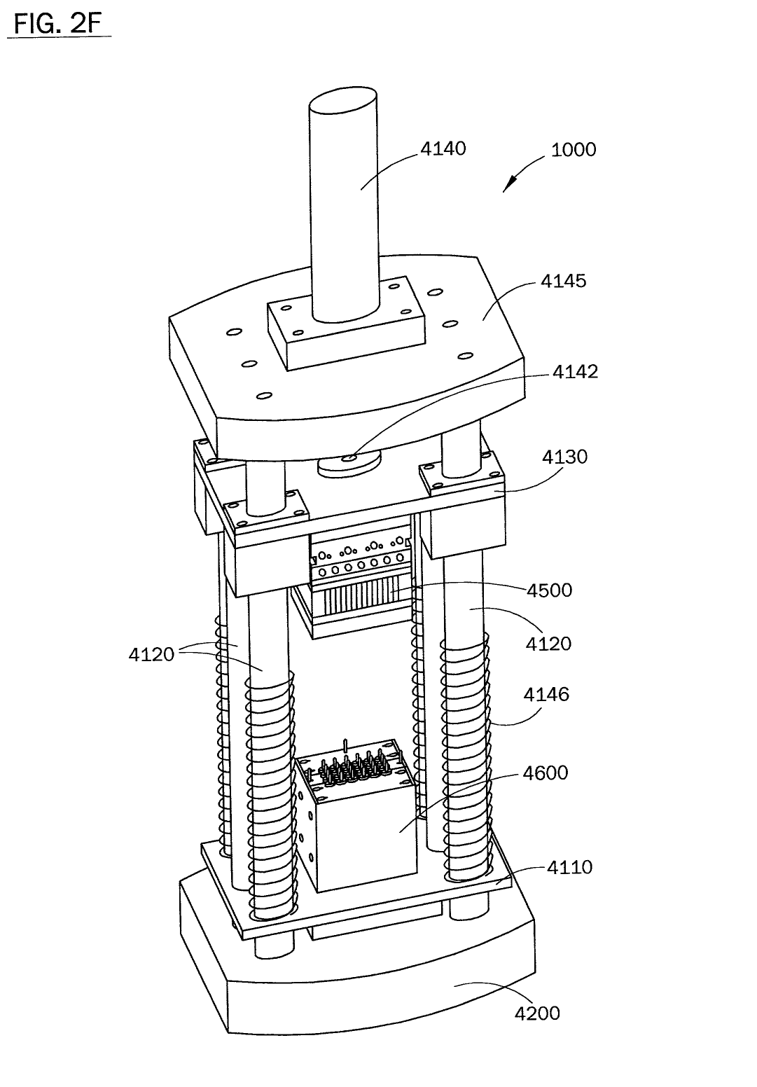

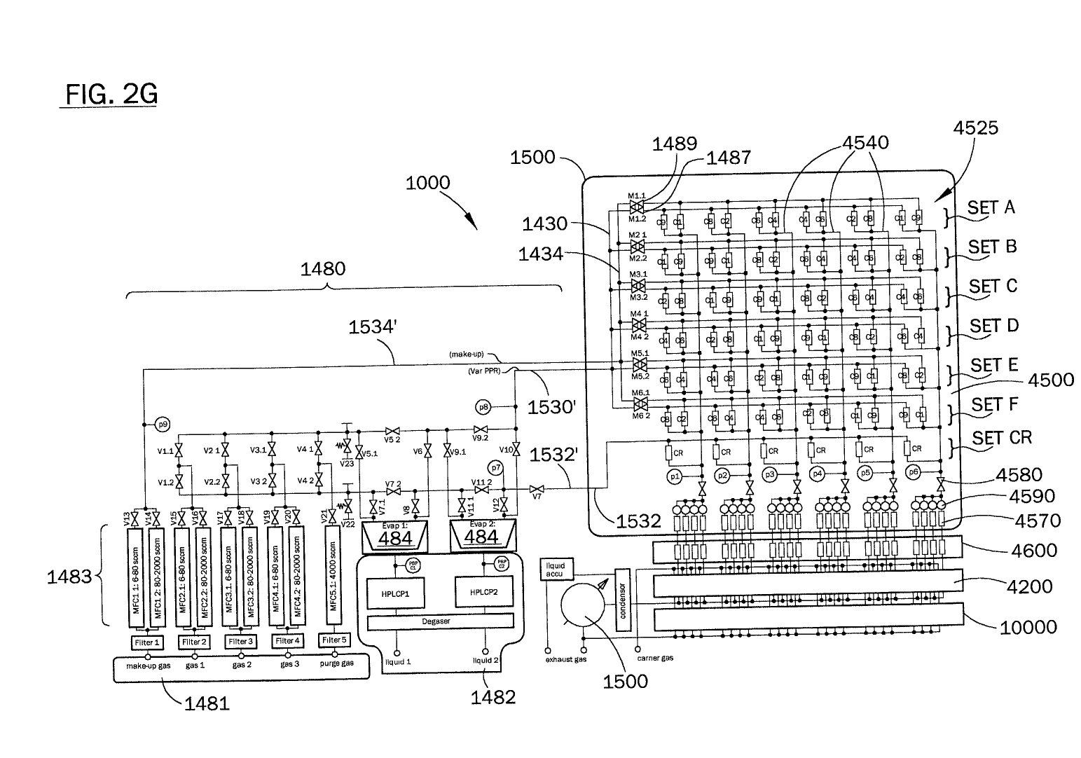

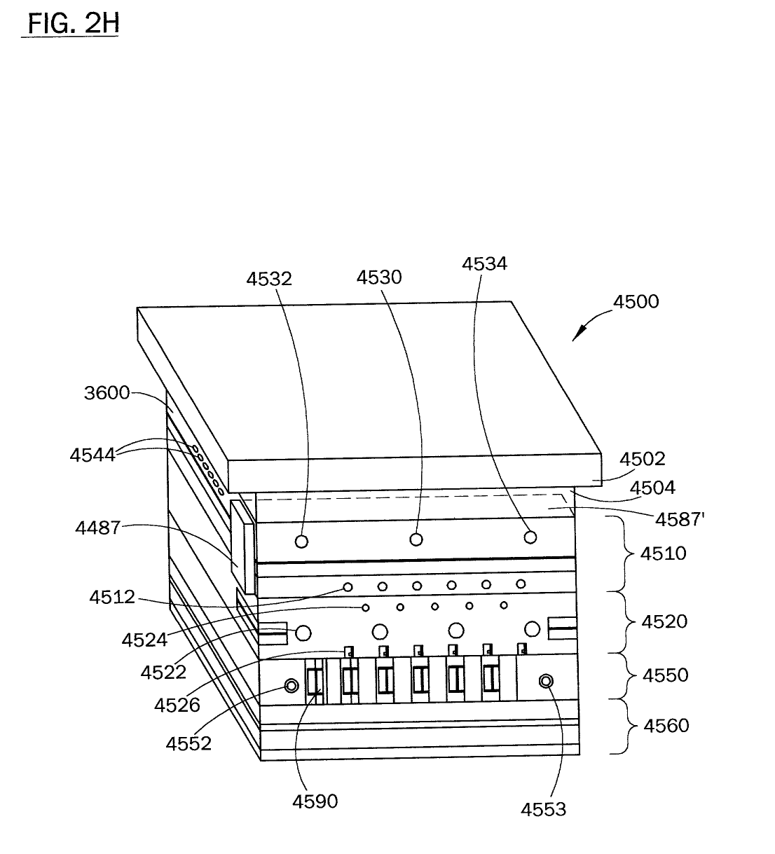

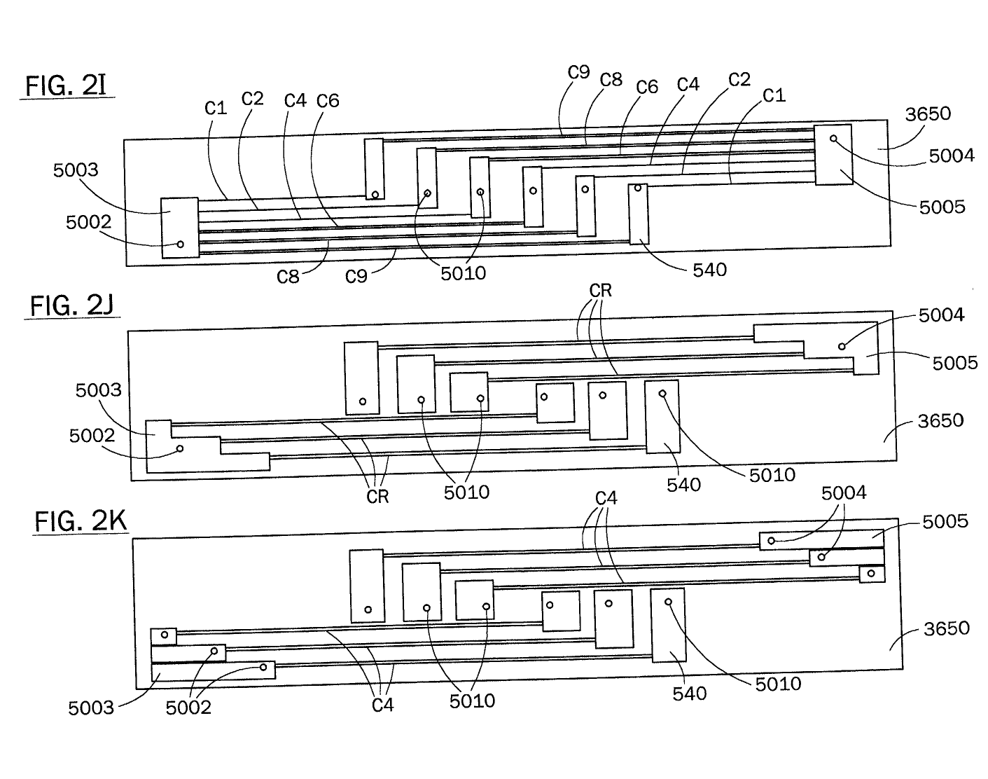

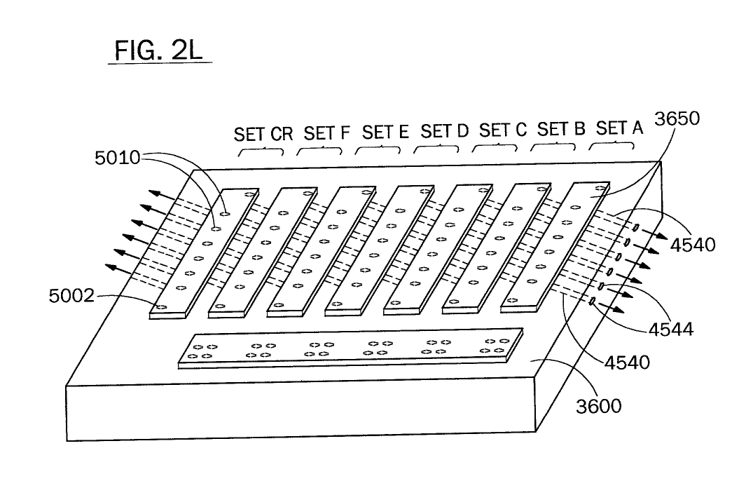

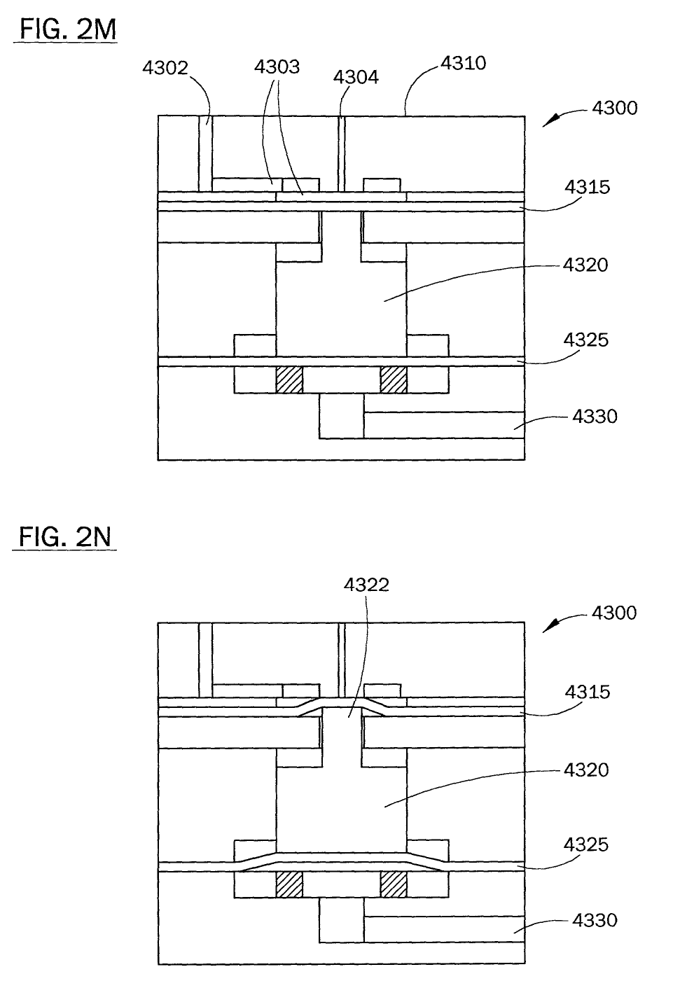

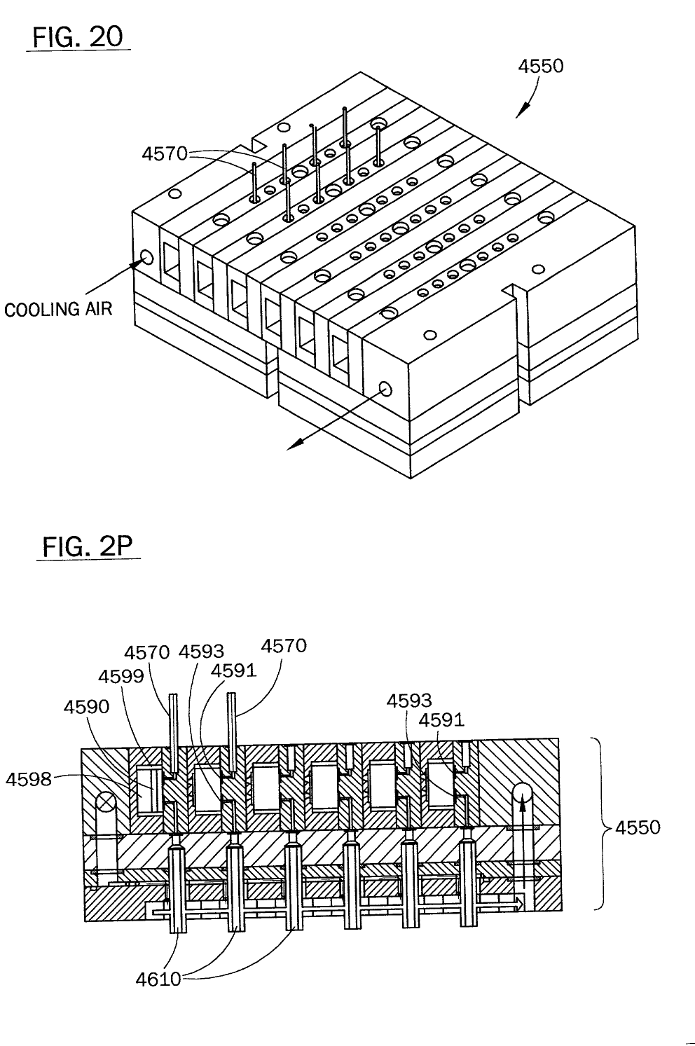

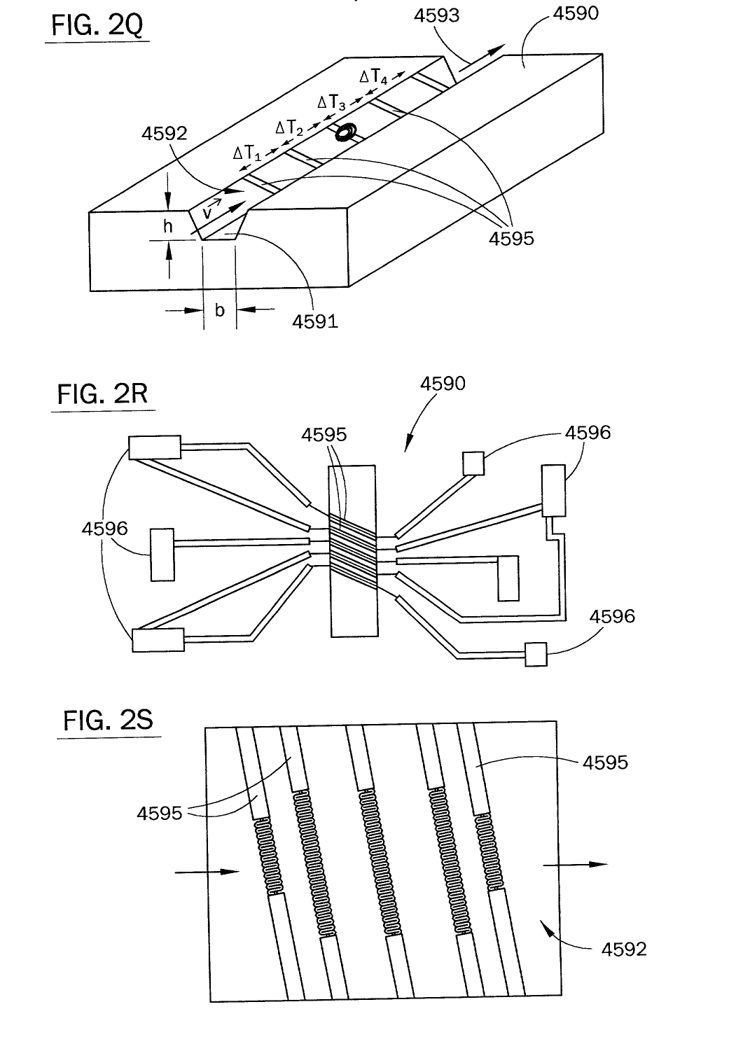

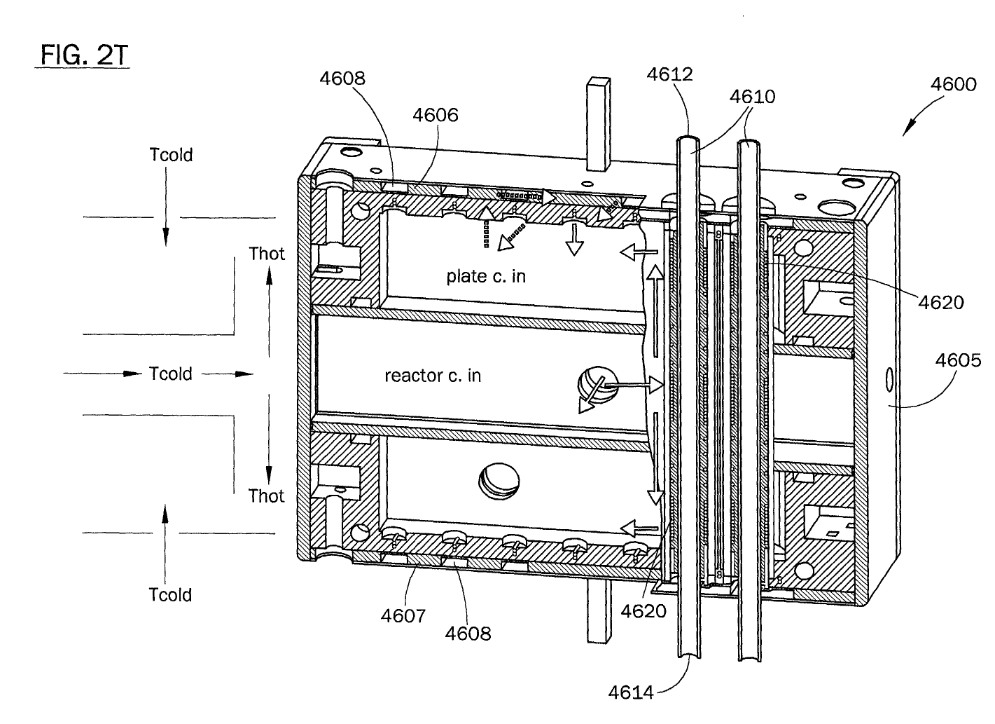

The present invention involves protocols for designing and implementing sets of simultaneous experiments, in a parallel, multi-variable process optimization reactor. The multi-variable process optimization reactor is preferably a parallel flow reactor having the operational capability to simultaneously and controllably vary reaction conditions between reaction vessels—either modularly or independently. The simultaneously varied reaction conditions preferably include at least two of the following, in various combinations and permutations: space velocity, contact time, temperature, pressure and feed composition. Compositional variations in the catalysts residing in each of the reaction vessels can also be investigated in the set of simultaneous experiments implemented in the parallel reactor. A preferred multi-variable process optimization reactor is disclosed in co-owned, co-pending U.S. patent application Ser. No. 60/185,566 filed Mar. 7, 2000 by Bergh et al., U.S. Ser. No. 09/801,390 filed Mar. 7, 2001 by Bergh et al., U.S. Ser. No. 09/801,389 filed Mar. 7, 2001 by Bergh et al., and U.S. Ser. No. 09/801,430 filed Mar. 7, 2001 by Srinivasan et al, each of which are incorporated by reference in its entirety for all purposes. Some aspects of this preferred parallel multi-variable optimization reactor are discussed below, in connection with FIGS. 2F through 2T.

Significantly, as described more fully below, the investigation of various reaction conditions for a reaction of interest in a simultaneous set of parallel experiments (e.g., in a single run through a parallel multi-variable process reactor) provides sufficient data for creating a meaningful master curve of determined selectivity for the reaction of interest versus determined conversion—for each of one or more catalyst compositions being investigated in that single run of parallel experiments. The obtention of sufficient data from a single set of simultaneous experiments to generate a meaningful master curve represents a substantial advance in the art of catalysis research, and particularly, heterogeneous catalysis research.

The simultaneously varied reaction conditions for the set of simultaneous experiments preferably include a combination of at least two reaction conditions: (i) either different space velocities or different contact times, in combination with (ii) either different temperatures, different pressures or different feed compositions. In some embodiments, the simultaneously varied reaction conditions for the set of simultaneous experiments preferably include different space velocities, in combination with different contact times. More specifically, preferred sets of simultaneously varied reaction conditions include: space velocity and temperature; space velocity and pressure; space velocity and feed composition; contact time and temperature; contact time and pressure; contact time and feed composition; and space velocity and contact time. In other embodiments, higher numbers of reaction conditions, such as three or more reaction conditions or four or more reaction conditions can be simultaneously varied in the set of simultaneous experiments. For example, the simultaneously varied reaction conditions can include one or more of the following preferred tertiary combinations of reaction conditions: different space velocities, temperatures and pressures; different space velocities, temperatures and feed compositions; different space velocities, pressures and feed compositions; and different temperatures, pressures and feed compositions. A preferred approach for simultaneously varying four different reaction conditions can include a combination of either: different space velocities, temperatures, pressures and feed compositions, or different contact times, temperatures, pressures and feed compositions.

Generally, the terms used herein to describe the various reaction conditions, including temperature, pressure, space velocity, contact time and feed composition have their ordinary meaning as understood by a person of ordinary skill in the art. Temperature and pressure are independent variables, whereas space velocity, contact time, and feed composition are partially coupled variables (i.e., two of the three can be independently controlled). Briefly, contact time is a measure of the total average time of exposure between a fluid and a catalyst in a flow reactor, and is typically defined as the volume of the reaction zone divided by the total volumetric flowrate of the feed. Space velocity is a measure of the molar, mass or volume flowrate of the reactant(s) or key reactant—and an be controlled independently of the total molar flowrate of feed (e.g., due to differences in concentration of the reactant(s) in the feed, such as due to dilution of reactants with one or more inert fluids). Feed composition refers to a measure of the relative ratio of various reactant components and/or inert components of the feed mixture. A more detailed discussion of these reaction condition parameters is provided in many texts. See, for example, Wijngaarden et al., “Industrial Catalysts—Optimizing Catalysts and Processes”, Wiley-VCH, Germany (1998).

In preferred protocols for any of the aforementioned embodiments, at two different space velocities, and preferably at least three or at least four or at least five or at least six different space velocities are simultaneously investigated in combinations with the other reaction condition(s) as described. Similarly, at least two, preferably at least three or at least four or at least five or at least six different temperatures are simultaneously investigated. Likewise, other reaction conditions (pressures, feed compositions, contact times) are simultaneously investigated using at least two, preferably at least three or at least four or at least five or at least six different values of such other reaction conditions. Particularly preferred combinations of reaction conditions to be simultaneously evaluated in a single set of parallel experiments include at least three space velocities with at least two temperatures, pressures, feed compositions or contact times. In some embodiments, at least six space velocities are evaluated simultaneously with at least four temperatures, pressures, feed compositions or contact times.

Generally, with reference to FIG. 2A, a method for evaluating process conditions for a catalyzed chemical reaction in a parallel flow reactor comprises simultaneously supplying one or more reactants through a fluid distribution system to each of six or more reactors of a parallel flow reactor under reaction conditions to effect a chemical reaction of interest, while controllably varying one or more sets of reaction conditions between each of the six or more reactors. A reactor effluent containing one or more reaction products and, in some cases, one or more unreacted reactants is simultaneously discharged from each of the six or more reactors. Each of the reactor effluent streams can be sampled and analyzed, preferably simultaneously analyzed, to determine the conversion of one or more of the reactants, and the selectivity for at least one reaction product for the reaction of interest. The conversion is preferably determined for the conversion-limiting reactant. Generally, as used herein, conversion refers to a measure of the extent to which a reaction has proceeded, and is typically defined as the fractional amount of the amount of reactant that has been converted to one or more products (regardless of whether the products are of interest, or are side reaction products). The selectivity refers to a measure of the extent to which a reaction resulted in a desired product of interest, and is typically defined as a ratio of the amount (e.g., molar amount) of desired product obtained to the amount (e.g., molar amount) of the key reactant converted. A more detailed discussion of conversion and selectivity is provided in Wijngaarden et al., “Industrial Catalysts—Optimizing Catalysts and Processes”, Wiley-VCH, Germany (1998).

Each of the six or more reactors comprises a catalyst having activity for the chemical reaction of interest. In general, depending on the number of reaction vessels in the parallel reactor, at least two or more, preferably at least four or more, and in some embodiments at least six or more of the catalysts are substantially the same—such that they have substantially the same composition and/or were prepared by substantially the same synthesis protocols with substantially the same compositional recipe and/or were prepared by substantially the same mechanical (e.g., grinding, pressing, crushing, sieving) treatments, chemical treatments, and/or physical treatments.

In preferred embodiments, the one or more sets of reaction conditions are controllably varied such that a determined conversion (e.g., the conversion of the conversion-limiting reactant) and a determined selectivity for one or more reaction products of the reaction of interest includes at least two, preferably at least three, preferably at least four, more preferably at least five and most preferably at least six data values for each of the catalyst compositions being evaluated. The determined data values for conversion preferably span a range of values that relate to the conversion range of interest for the chemical reaction, such that a meaningful master curve can be generated for each of the catalyst compositions being evaluated. Generally, the determined conversion values include four or more values, preferably six or more values, that span a range of at least about 10% conversion difference, and more preferably at least about 20% conversion difference between the highest and lowest of such values, and for many reactions of interest, more preferably at least about 30%, 40%, 50% or 60% conversion difference between the highest and lowest of such values. Considered in another manner, the determined conversion values (e.g., of the conversion-limiting reactant) include four or more values, preferably six or more values ranging from less than about 20% conversion to more than about 40% conversion. That is, within the determined three or six values for conversion, at least one of the determined values is less than about 20% conversion, and at least one of the determined values is more than about 40% conversion. Preferably, the set of reaction conditions are varied in the single set of simultaneous parallel reactions such that the determined conversion values (e.g. of the conversion-limiting reactant) include a range of four or more values, preferably six or more values, ranging from less than about 15% conversion to more than about 45% conversion, preferably ranging from less than about 10% conversion to more than about 50% conversion, and in some embodiments, ranging from less than about 10% conversion to more than about 70% conversion, or even ranging from less than about 10% conversion to more than about 80% conversion. For some reactions of interest (e.g., propylene oxidation to propylene oxide; e.g., benzene to aniline conversions), relatively lower conversions are commercially significant due to economics, thermodynamics and/or safety considerations, typically with relatively higher selectivities. Hence, for some reactions of interest, the determined conversion values (e.g., of the conversion-limiting reactant) include three or more values, preferably four or more values, more preferably six or more values ranging from about 2% or less to more than about 5%, preferably from about 1% or less to more than about 10% conversion, more preferably from about 1% or less to more than about 15%. That is, within the determined three or six values for conversion, at least one of the determined values is about 2% or less (preferably about 1% or less), and at least one of the determined values is more than about 5% conversion (preferably more than about 10%, more preferably more than about 15% conversion).

Preferred Evaluation Protocols

In one preferred embodiment, a parallel reactor having six or more reaction vessels is loaded with a set of six or more of substantially the same catalysts (e.g. having substantially the same composition). A set of reaction conditions is controllably varied between the six or more reaction vessels, with the particular combinations of varied reaction conditions being selected generally from those described above. Preferably, with reference to FIG. 2B, the set of reaction conditions varied between the six or more reactors (having substantially the same catalyst, indicated as “C1” in FIG. 2B) include (i) at least three different space velocities (SV1, SV2, SV3) or alternatively, at least three different contact times (CT1, CT2, CT3), and (ii) at least two different temperatures (T1, T2), or alternatively, at least two different pressures (P1, P2), or alternatively, at least two different feed compositions (FC1, FC2). As shown in FIG. 2B, this set of parallel experimental conditions can be represented schematically in 3×2 matrix with the catalyst (C1) indicated in each box (e.g., such that each box represents a separate reaction vessel of the parallel flow reactor) and with the reaction conditions applied to the catalyst-containing reactors being indicated along each side of the matrix.

In a variation of the immediately preceding embodiment, a parallel reactor having twelve or more reaction vessels is loaded with a set of twelve or more of substantially the same catalysts (e.g. having substantially the same composition). Two sets of reaction conditions are controllably, and independently varied between six or more reaction vessels per set—a first set of reaction conditions being varied between a first set of six or more reactors, and a second set of reaction conditions being varied between a second set of six or more reactors, with the particular combinations of varied reaction conditions being selected generally from those described above. Preferably, with reference to FIG. 2C, the first set of reaction conditions varied between the first set of six or more reactors (e.g., having catalyst C1) include (i) at least three different space velocities (SV1, SV2, SV3) or alternatively, at least three different contact times (CT1, CT2, CT3), and (ii) at least two different temperatures (T1, T2). The second set of reaction conditions varied between the second set of six or more reactors (e.g., having catalyst C1) include (i) at least three different space velocities (SV1, SV2, SV3) or alternatively, at least three different contact times (CT1, CT2, CT3), and (ii) at least two different pressures (P1, P2), or alternatively, at least two different feed compositions (FC1, FC2). The space velocity and/or contact time can be the same or different as compared between the first set of reaction vessels and the second set of reaction vessels. As shown in FIG. 2C, this set of parallel experimental conditions can be represented schematically in 3×4 matrix.

In another preferred embodiment, a parallel reactor having twelve or more reaction vessels is loaded with twelve or more catalysts—each having activity for the chemical reaction of interest. A first set of the twelve or more catalysts can be six or more first catalysts that are substantially the same (e.g. having substantially the same first composition). A second set of the twelve or more catalysts can be six or more second catalysts that are substantially the same (e.g. having substantially the same second composition). A first set of reaction conditions is controllably varied between the reaction vessels comprising the first set of catalysts, with the particular combinations of varied reaction conditions being selected generally from those described above. Similarly, and simultaneously therewith, a second set of reaction conditions is controllably varied between the reaction vessels comprising the second set of catalysts, with the particular combinations of varied reaction conditions being selected generally from those described above. Preferably, with reference to FIG. 2D, the first set of reaction conditions, and the second set of reaction conditions are varied between their respective reaction vessels to include (i) at least three different space velocities (SV1, SV2, SV3) or alternatively, at least three different contact times (CT1, CT2, CT3), and (ii) at least two different temperatures (T1, T2), or alternatively, at least two different pressures (P1, P2), or alternatively, at least two different feed compositions (FC1, FC2). As shown in FIG. 2D, this set of parallel experimental conditions can be represented schematically in 3×4 matrix.

In a further preferred embodiment, a parallel reactor having sixteen or more reaction vessels is loaded with sixteen or more catalysts—each having activity for the chemical reaction of interest. The sixteen or more catalysts include at least four sets of catalysts, each set having four or more catalysts. The catalysts within each of the four sets are substantially the same (e.g., or at least having substantially the same composition), but the catalysts as compared between different sets vary with respect to each other. Specifically, the sixteen or more reactors can comprise a first set of four or more of the catalysts having substantially the same first composition, a second set of four or more of the catalysts having substantially the same second composition, a third set of four or more of the catalysts having substantially the same third composition, and a fourth set of four or more of the catalysts having substantially the same fourth composition. A set of reaction conditions is controllably varied between the sixteen or more reaction vessels—with the particular combinations of varied reaction conditions being selected generally from those described above. Preferably, with reference to FIG. 2E, the set of reaction conditions are varied between to include (i) at least four different space velocities (SV1, SV2, SV3, SV4) or alternatively, at least four different contact times (CT1, CT2, CT3, CT4), and (ii) at least four different temperatures (T1, T2, T3, T4), or alternatively, at least four different pressures (P1, P2, P3, P4), or alternatively, at least four different feed compositions (FC1, FC2, FC3, FC4). Additionally, the set of reaction conditions are varied such that at least one catalyst from each of the first, second, third and fourth sets of catalysts sees each of the reaction conditions (considered independently, such that not all catalysts see all of the possible combinations of conditions). That is, the set of reaction conditions are varied such that at least one catalyst from each of the first, second, third and fourth sets of catalysts that catalyzes the chemical reaction under each of the at least four different space velocities or contact times, and under each of the at least four different temperatures, pressures or feed compositions. As shown in FIG. 2E, this set of parallel experimental conditions can be represented schematically in 4×4 matrix. In a variation of this approach, the number of reaction conditions being varied can increase for evaluation of the four or more sets of different catalysts. Specifically, for example, the parallel flow reactor can comprise twenty-four or more reactors to effect the chemical reaction of interest, with each of the twenty-four or more reactors comprising a catalyst having activity for the chemical reaction. The varied set of reaction conditions can comprise (i) at least six different space velocities or contact times, and (ii) at least four different temperatures, pressures or feed compositions, and the set of reaction conditions can be varied such that at least one catalyst from each of the first, second, third and fourth sets of catalysts catalyzes the chemical reaction under each of the at least six different space velocities or contact times, and under each of the at least four different temperatures, pressures or feed compositions, although not necessarily under each permutation of combinations of such reaction conditions.

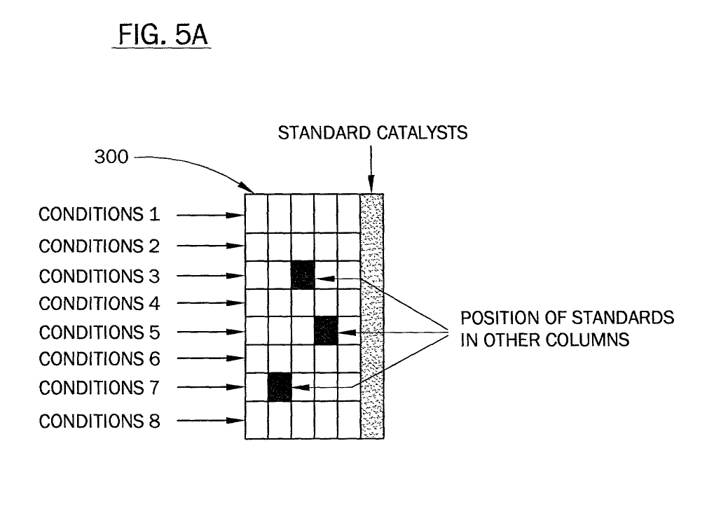

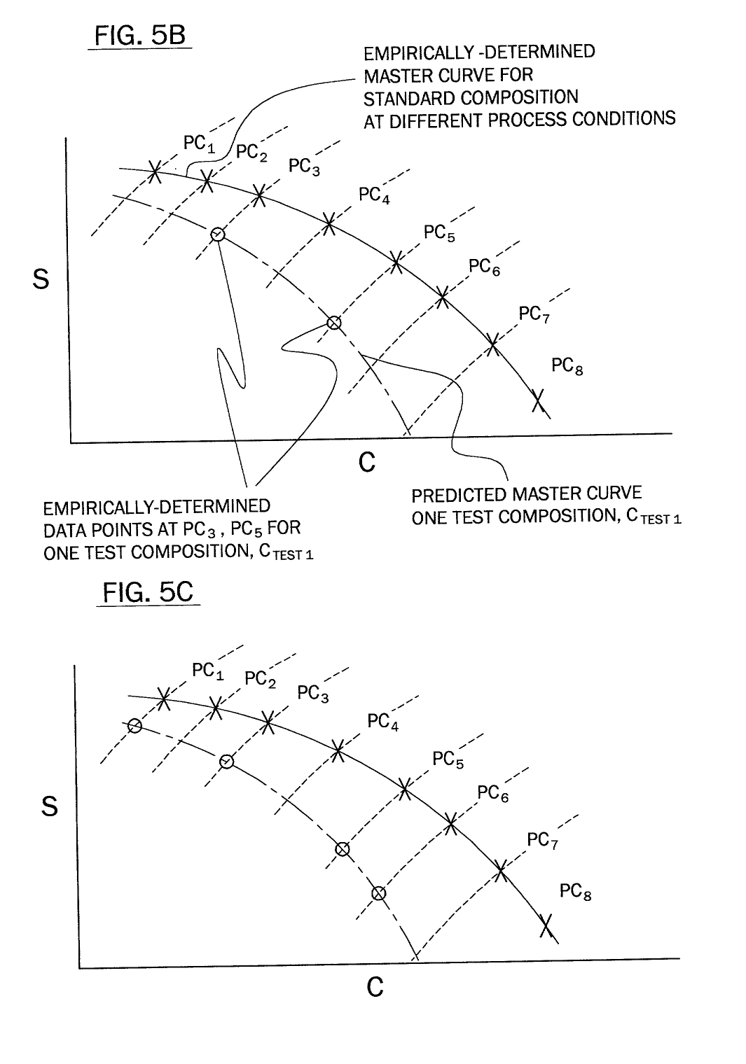

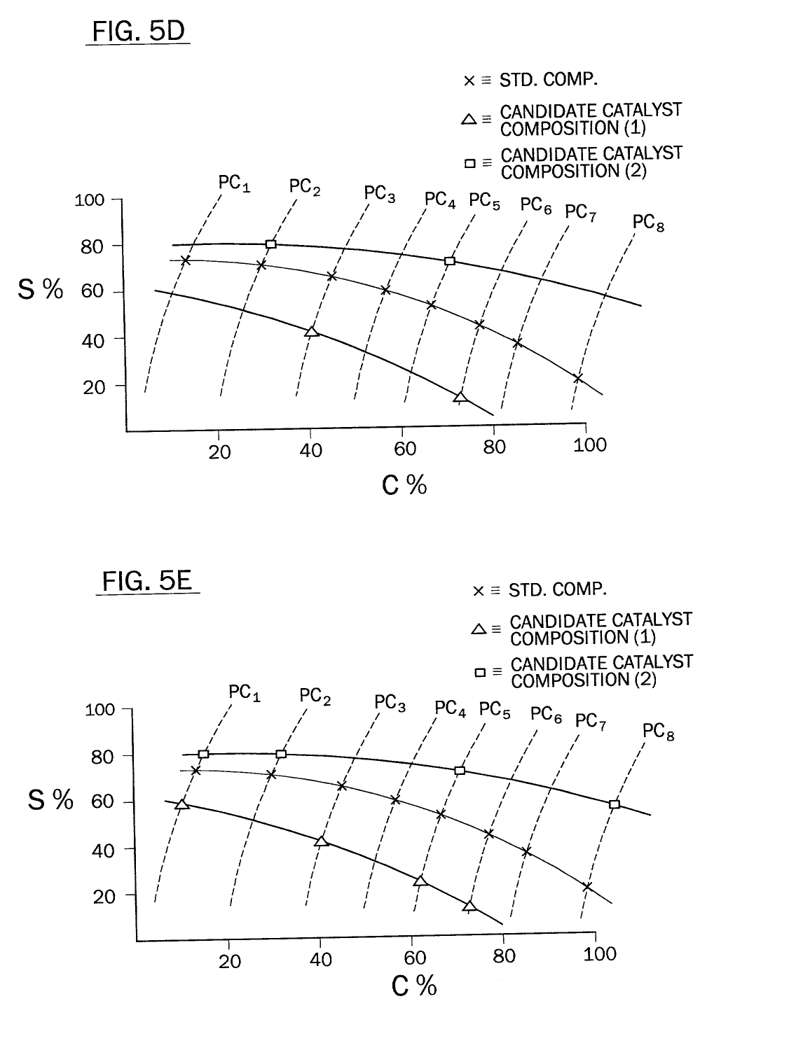

Advantageously, the immediately preceding experimental protocol for a multi-channel, multi-variable set of simultaneous experiments provides sufficient data to generate a meaningful master curve, without performing as many experiments as would be necessary to provide data for each of the four or more compositions at each particular combination of reaction conditions. The utility of this approach can be enhanced when combined with one or more interpolation and/or extrapolation techniques, such as predictive extrapolation based on a defined similarities with a related reference composition. To this end, in one variation of the embodiment described immediately above, the parallel flow reactor can comprises twenty or more, preferably twenty-four or more reactors of a parallel flow reactor to effect the chemical reaction of interest, each of the twenty or more, preferably twenty-four or more reactors comprising a catalyst having activity for the chemical reaction of interest. Four or more, and preferably eight or more of the reactors can comprise a reference set of four or more, preferably eight or more reference catalysts having substantially the same reference composition. The reference composition can be a standard composition that is a representative composition for the four or more sets of different catalysts (C1, C2, C3, C4) being evaluated. As a non-limiting example, the reference composition can be the same catalyst platform and have the same major components (e.g., components having a relative molar ratio of more than about 10%) as one or more of the other catalysts being evaluated, but different minor components (e.g. dopants) having a relative molar ratio of 10% or less). As another non-limiting alternative, the reference composition can be the same catalyst platform and have the same or functionally similar components with a relative molar ratio of components that is about the average of the range of molar ratios being evaluated in the library. In any case, the reference composition is repeated at least four times, preferably at least eight times, and the reaction conditions to which the reference compositions are exposed are preferably varied in the same manner as they are varied for the four or more sets of different catalysts, such that the four or more, preferably eight or more see at least four or more, preferably eight or more of the varied reaction conditions or combinations thereof. For at least one, and preferably for each of, the first, second, third or fourth catalyst compositions, at least a portion of the master curve can be interpolated or extrapolated by comparison with a master curve determined for the eight or more reference catalysts. The comparative interpolation and/or extrapolation can be visual or qualitative, and/or can be based on mathematical and statistical modeling approaches known in the art. In addition to interpolation and/or extrapolation between determined data points based on a master curve for the reference catalyst, one can also interpolate and/or extrapolate entire master curves (or substantial portions thereof) based on comparison to master curves developed for similar catalysts using one or more of the approaches described herein. Further details about such a predictive approach are described below in connection with FIGS. 5A through 5E.

In any of the aforementioned embodiments, additional blank reaction channels—having an essential absence of catalytic activity for the reaction of interest—can also be employed, for example, to determine background and/or to detect reaction conditions that may decompose reactants or products.

In yet a further application, a parallel process optimization reactor can be used to effect a single set of simultaneous (parallel) experiments to check for diffusion limitations, useful for example in screens directed toward determining intrinsic activity and/or kinetic activity of a catalyst composition. Briefly, a parallel reactor having twelve or more reaction vessels is loaded with a set of twelve or more of substantially the same catalysts (e.g. having substantially the same composition). Two sets of reaction conditions are controllably, and independently varied between six or more reaction vessels per set—a first set of reaction conditions being varied between a first set of six or more reactors, and a second set of reaction conditions being varied between a second set of six or more reactors. Specifically, the varied first set of reaction conditions generally comprises varied average particle size of the catalysis materials, prepared, for example, as described in co-owned, co-pending, U.S. patent application Ser. No. ______ entitled “Methods and Apparatus for Mechanical Treatment of Materials Such as Catalysts” filed on the date even herewith by Lugmair et al. The varied second set of reaction conditions can include linear gas flowrates through a reaction zone (e.g., through a fixed bed of a fixed bed reactor). Preferably, the varied first and second sets of reaction conditions can be employed in conjunction with a substantially constant space velocity and/or contact time. For example, increased gas flow can be realized without substantial effect on space velocity and/or contact time if the catalyst mass and bed height are also increased, or if a diluent (e.g., SiC) is employed, by varying the amount of the diluent. By varying particle size in six or more reaction vessels, and by varying linear (gas) reactant flowrate in six or more vessels, one can evaluate whether film diffusion and/or pore diffusion are limiting. The diffusion-limited nature of a reaction (or the lack of a diffusion-limited nature) is helpful in evaluating intrinsic activity or kinetic activity.

As noted, although described herein in connection with heterogeneous catalysis research with a parallel flow reaction system, the present invention has applications to homogeneous reaction systems, and for both heterogeneous and homogeneous catalysts, to parallel semi-continuous reaction systems or to parallel batch reaction systems. Furthermore, it is contemplated that other specific variations and combinations of parameters in a multidimensional space can be employed. See, for example, U.S. patent application Ser. No. 60/198,208 entitled “Combinatorial Parameter Space Experiment Design” filed Apr. 19, 2000 by Falcioni et al, which is hereby incorporated by reference in its entirety for all purposes.

Additional details about the preferred reaction system, about combinatorial catalysis research approaches, about preferred candidate materials and libraries of candidate materials, about preferred reactions of interest, and about various alternative approaches for evaluating catalysts—for use in connection with the above-described protocols—are described below.

Parallel Multi-Variable Process Optimization Reactor

A preferred parallel, multi-variable optimization reactor suitable for use in connection with the methodologies of the invention is shown in FIGS. 2F through 2T. With reference to FIG. 2F, the twenty-four channel reaction system 1000 is a bench-top instrument comprising a distribution module 4500 and a reactor module 4600. The distribution module 4500 and reactor module 4600 are supported in a frame 4100 that includes a lower support shelf 4110, and upper support shelf 4130 and four guide posts 4120 (linear ball bearings). The distribution module 4500 can be operationally engaged with the reactor module 4600 by downward movement of a shaft 4142 connected at a lower end to the distribution module 4500 via a support block (not shown), and at an opposing upper end to a hydraulic jack 4140 (e.g. 100 kN, adjustable, regulated force) supported on a press frame 4145. Preloaded springs 4146 are provided on each of the guide posts 4120 to ensure appropriate seating force between the reactor module 4600 and the lower support shelf 4110 to sequentially demount the distribution module from the reactor module (release of upper o-ring seals on reactor module), and subsequently, the reactor module lower o-rings). A gas-chromatograph-connector module 4200 provides twenty-four channel fluidic interface to a parallel gas chromatograph injection valve (not shown) situated under the lower support shelf 4110, and is in fluid communication with the reactor module 4600.

Referring to FIG. 11G, the distribution module 4500 can be interfaced with an external fluid distribution system 1480 having gas supply system 1481 with associated mass flow controllers 1483, and a liquid supply system 1482 with associated evaporators 1484, together with appropriate valving, substantially as described above. Three gaseous feed components are fed through external transfer lines 1530′ (variable partial pressure reactant feed source), 1532′ and (constant partial pressure reactant feed source), 1534′ (make-up gas) substantially as shown to an internal inlet fluid distribution system 1500 having an internal feed-composition subsystem 4525. Internal feed transfer lines 1530, 1534, and pairs of commonly-actuated inlet isolation valves 1487, 1489 provide selectable fluid communication to a series of six sets of flow restrictor groups—indicated as SET A, SET B, SET C, SET D, SET E, and SET F—with each set comprising six groups of flow restrictors. Each group of flow restrictors in a particular set include a first-feed-component flow restrictor in selectable fluid communication with the variable partial pressure feed source through transfer line 1530 and inlet isolation valve 1487, and a second-feed-component flow restrictor in fluid communication with the make-up feed source through transfer line 1534 and inlet isolation valve 1489. The relative conductance values for the first-feed-component flow restrictor and the second-feed-component flow restrictor within each group is indicated as “C1”, “C2”, “C4”, “C6”, “C8” and “C9”, with combinations of values rotating between the various groups as appropriate. In one configuration, internal feed transfer line 1532 provides a constant partial pressure feed source through a set of six dedicated flow restrictors, where the flow restrictors have substantially the same resistance to flow (i.e., “constant resistance” flow restrictors, indicated as SET CR). The (varied) feed composition from the six groups of feed-component flow restrictors (after mixing feed components from the first-feed-component flow restrictors and second-feed-component flow restrictors in an internal mixing zone, not shown,) are fed through six respective discharge channels 4540. The combined feed composition (of the variable feed component/make-up feed component) are mixed with the constant feed component in the discharge channels 4540. A pressure sensor (illustrated as p1, p2, p3. etc.) monitors discharge channel 4540 pressure. Each of the resulting six varied feed composition feeds are discharged through an outlet isolation valve 4580, and then split four ways using flow-splitters to create twenty-four streams—with six groups of four steams each, and each group having a varied feed composition. Each of the twenty-four feed streams can be fed through a single, individual split restrictor 4570—such as a capillary or as microfluidic channel (e.g. having substantially the same resistance to flow as compared between reaction channels), and then through a mass flow sensor 4590 (MFS), before leaving the distribution module. As illustrated, the order of the mass flow sensor 4590 and the split restrictor 4570 can be reversed from that described above. The distribution module 4500 is a separate, stand-alone subsystem that mounts in fluid communication with the reactor module 4600. Connector module 4200 and detection module 10000 are also shown.

The distribution module 4500 was provided as a modular unit comprising a plurality of modular fluidic chips. In particular, with reference to FIG. 2H, the distribution module comprises a flow-restrictor block 4510 comprising a substrate 3600 having several microchip bodies (not shown) mounted thereon. The microchip bodies include flow restrictors (C1, C2, C3, etc.) from which sets of flow-restrictor groups are formed by fluidic connection. A series of commonly-actuated groups of microvalves can be precision machined and mounted as a valve block 4487 on one side of the distribution module 4500, or integrally therewith, including for example as a subblock integrally within the flow-restrictor block 4510—for example, as indicated as 4587′ with dotted lines. Alternatively, the groups of microvalves can be microfabricated and be integral with the flow restrictor block 4510 or with microchip bodies mounted on the flow-restrictor block 4510. The microvalves are pneumatically actuated with air supplied through control pressure ports 4512. Reactant sources, including a variable partial pressure gas, a make-up gas and a constant partial pressure gas are supplied via external-internal inlet ports 4530, 4534, 4532, to provide fluid connection with internal transfer lines 1530, 1534, 1532, respectively (FIG. 2G). A cover 4504 and an insulator block 4502 are positioned over the flow-restrictor block 4510. The flow-restrictor block 4510 is situated over a heater block 4520 comprising resistive cartridge heaters 4522, pressure-sensor ports 4524 and thermocouple ports 4526. Capillary-type split restrictors (4570, FIG. 2G) extend between the heater block 4520 and the split-restrictor-mass flow sensor block 4550 (“SR-MFS block” 4550), that includes an array of microfabricated mass flow sensors 4590 downstream from each of the split restrictors, and connection ports 4552, 4553 for cooling fluid circulating through the SR-MFS block 4550. The feed passes from the mass flow sensors into reactor tubes (not shown) that extend upward from the reactor module (4600, FIG. 2G) into a press block 4560 when the distribution module 4500 is engaged with the reactor module.

The flow restrictor block 4510 includes six sets of flow-restrictor-groups microfabricated on separate microchip bodies 3650 that are mounted on a common substrate 3600. In one embodiment, a set of seven microchip bodies having integral flow restrictors can be mounted on a substrate, with each the flow restrictors in each of the mircochip bodies corresponding to one of the sets of flow-restrictor groups (SET A, SET B, SET C, SET D, SET E, SET F, SET CR) represented in FIG. 2G. Exemplary microchip bodies, corresponding to SET A and SET CR are shown in FIG. 21 and 2J, respectively. The microchip bodies comprise a first inlets 5002 (e.g. in fluid communication with one of the feed-component source gasses, such as the variable-feed component), and a second inlet 5004 (e.g., in fluid communication with another of the feed-component source gas, such as the make-up feed component). When a particular set of flow restrictors is selected by actuation of microvalves (1487, 1489, FIG. 2G) corresponding to that set, then the two feed components come in through the inlets 5002, 5004 into the inlet plenums 5003, 5005, and flow through the flow restrictors (e.g., for SET A, varied flow restrictors C1, C2, C4, C6, C8, C9; for SET CR, constant restrictors CR) to mixing zones 1540, such that the varied feed compositions having various ratios of feed components are formed in the mixing zones. Microfluidic outlets 5010 provide fluid communcation to the discharge (channels 4540, FIG. 2G). As an alternative, geometry effects associated with variously-sized channels off of the common inlet plunums 5003, 5005 (e.g. entrance volume effects) can be minimized physically forming, for example, the C9 flow restrictor from nine multiple, identical copies of the C1 flow restrictor. With reference to FIG. 2K, in a further embodiment, the flow resistances on each particular microchip body be substantially the same (e.g., C4, as illustrated, for one microchip body, C6 for another microchip body, etc.). Also, each flow restrictor (e.g., C4, as illustrated) can have its own, dedicated micrfluidic inlet 5002 and inlet plenum 5003. Referring to FIG. 2L, the sets of flow restrictors (with grouped resistance values as shown in FIG. 2G) can be established where each of the microchip bodies are fabricated to be identical (e.g., such as shown in FIG. 21)—without physically and integrally rotating the various combinations of flow resistances on the microchip body—by having microfluidic channels 4540 that cross the outlets 5010 of each flow restrictor (C1, C2, C4, etc.) in the various combinations, so that by selection of appropriate feed components to the inlets 5002, the desired sets of flow restrictors (e.g. SET A, SET B, SETC, etc.) can be achieved. Internal passages within the flow-restrictor block 4520 and/or cover block 4510, 4504 can be used for internal interconnections. Such internal passages can be supplemented by external interconnections that interface through side ports 4544. Alternatively, such side-ports could be manifolded and routed through a different face of the flow-restrictor block 4510. Fabrication of the flow restrictors integral with the microchip bodies can be effected, for any of the aforementioned embodiments, using typical microfabrication techniques.

The flow restrictor block 4510 also includes the six pairs of commonly-actuated inlet isolation microvalves 1487, 1489, as well as the outlet isolation valves 4580. These valves are preferably fabricated using precision machining techniques known in the art. Alternatively, the valves can be microfabricated, and can be integral with the flow-restrictor block 4510 or with a microchip body mounted thereon. The valves can also be, as noted above, part of an external fluid distribution system (1480, FIG. 2G). The particular microvalve design is not critical. Preferably, the microvalves 1487, 1489 are membrane-actuated, membrane-seated valves such as shown in FIGS. 2M and 2N. Briefly, membrane-actuated valves 4300 can be prepared by precision machining to form the various component parts. In its open state (FIG. 2M), a fluid can flow into the valve through fluid inlet passage 4302, through internal passages 4303, past the valve seat 4310, and out through outlet passage 4304. In its closed state (FIG. 2N), a piston 4320 having a piston face 4322 is forced upward against a seating membrane 4315 such that fluid flow past the seat 4310 is sealingly blocked, with the seating membrane 4315 essentially acting as a gasket between the piston face 4322 and the valve seat 4310. The piston 4320 is preferably pneumatically actuated by use of an actuating membrane 4325 under pressure through actuation passage 4330. Portions of the seating membrane 4315 and actuating membrane that are situated between facing component surfaces of the valve body can serve as gaskets when the valve is clamped or fastened together. Further details are provided in co-pending, co-owned application U.S. Ser. No. 60/274,022 entitled “Gas Chromatograph Injection Valve Having Microvalve Array” filed on Mar. 7, 2001 by Bergh et al.

The mass-flow-sensor/split restrictor block 4550 is shown in FIGS. 20 and 2P. The MFS-SR block 4550 comprises, for each of the twenty-four reaction channels, a split restrictor 4570, and a microfabricated mass-flow sensor 4590. As shown in FIG. 20, the unit is cooled using fluid-type micro heat exchanger with cooling air as the cooling medium. The split restrictor is preferably a capillary-type flow restrictor (e.g., 1/16 O.D. /125 μm I.D. stainless steel capillaries). For each reaction channel, the feed gas flows through the split restrictor into the mass-flow sensor 4590 via sensor inlet 4591. The feed gasses passes through a detection channel 4592 and then exits via sensor outlet 4593. O-rings 4599 are used to seal the sensor inlet and outlet. 4591, 4593. The mass-flow sensor design is not critical. Referring, briefly, to FIGS. 2Q, 2R, 2S, a preferred microfabricated mass flow sensor 4590 includes five-detection filaments 4595. The detection filaments 4595 are electrically connected to contact pads 4596 via conductive paths. The contact pads are connected to mass-sensor electronic circuitry for a five-bridge design, according to known techniques. The detection filaments 4595 are preferably platinum vapor-deposited onto a silicon nitride bridge, and designed with a meandering path (FIG. 2S). The silicon nitride bridge is positioned at 15° angle relative to the normal to the detection channel 4592. The electronic circuitry (e.g. printed circuit board) can be located adjacent each mass flow sensor (e.g., in the adjacent cavity 4598). After exiting the mass flow sensor 4590, the feed gas for each channel is fed to the reactor tubes 4610.

The reactor module 4600, shown schematically in FIG. 2T, comprises a 4×6 array of twenty-four reactor tubes 4610 individually supported in a reactor frame 4605. Each tube has a reaction volume of about 1 ml. Each of the reactor tubes 4610 can be individually heated using resistive coil heaters 4620 (e.g. Watlow Mini-K-ring). Thermal isolation between reactor tubes 4610 is achieved using fluid-type heat exchanger to cool the inter-reactor volume within the reactor frame 4610. Preferably, the cooling medium is air or inert gas, and is fed into the reactor module 4600 substantially at the midsection thereof. Plate cooling fluid (e.g. air) is also fed through the top member 4606 and bottom member 4607 of the reactor frame 4605, specifically through heat-exchange channels 4608 formed therein. Advantageously, as described in greater detail above, the heat flux associated with the resistive coil heaters 4620 can be axially varied to account for heat variations due to the reaction, and to balance heat removal by the cooling media such that a substantially axial uniform temperature profile is obtained. Further details about temperature control are provided in co-owned, co-pending application U.S. Ser. No. 60/274,065 entitled “Parallel Flow Reactor Having Improved Thermal Control” filed on the date even herewith (Mar. 7, 2001) by Bergh et al. The feed gas flows into the reactor tube inlet 4612, and optionally contacts a catalyst (e.g., supported in the reactor tube using frits (not shown)) under reaction conditions to effect the chemical reaction of interest. The reaction products and unreacted reactants are discharged through the reactor tube outlet 4614.

Further details of this preferred reaction system are described in co-owned, co-pending U.S. patent application Ser. No. 60/185,566 filed Mar. 7, 2000 by Bergh et al., U.S. Ser. No. 09/801,390 filed Mar. 7, 2001 by Bergh et al., U.S. Ser. No. 09/801,389 filed Mar. 7, 2001 by Bergh et al., and U.S. Ser. No. 09/801,430 filed Mar. 7, 2001 by Srinivasan et al., each of which are incorporated by reference in its entirety for all purposes.

Combinatorial (i.e., High-Throughput) Catalysis Research

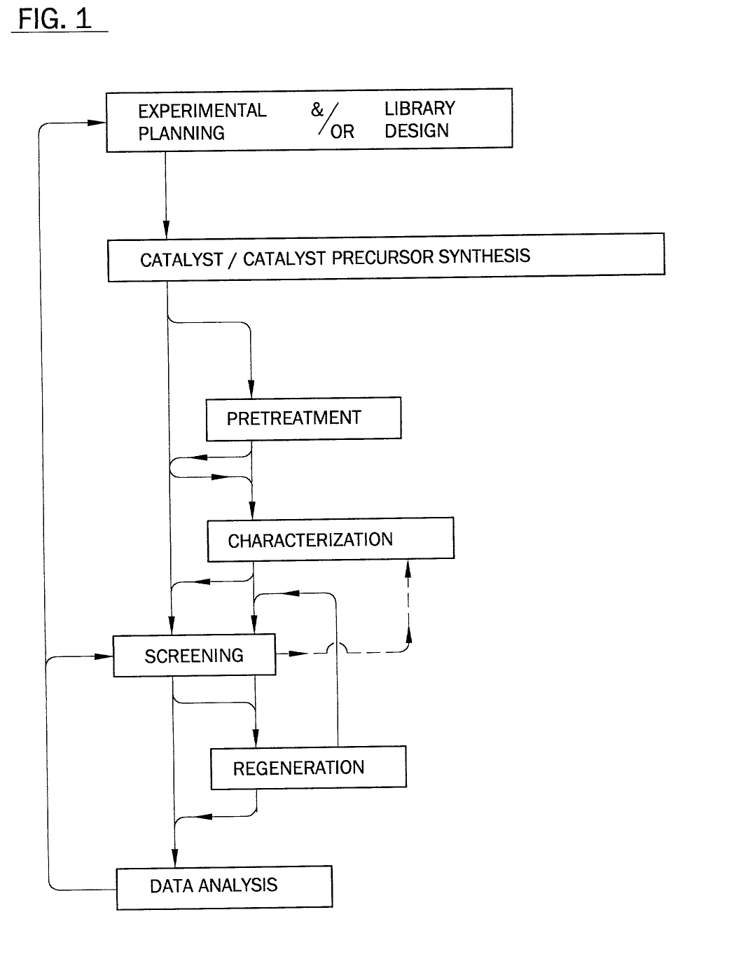

With reference to FIG. 1, major steps in a comprehensive combinatorial (i.e., high-throughput) research program for catalysis, and in particular, for heterogeneous catalysis can generally comprise one or more of the following steps:

1) Experimental Planning/Library Design

2) Synthesis of Catalyst or Catalyst Precursor Library

3) Optionally, Pretreatment of Catalyst or Catalyst Precursor Library

a) chemical treatment (e.g. precursor decomposition, oxidation, reduction, activation)

b) physical treatment (e.g., calcining)

c) mechanical treatment (e.g., pelletizing, grinding, sieving)

4) Optionally, Characterization of Catalyst or Catalyst Precursor Library (x-ray diffraction, infrared)

5) Screening (Reaction Based) of Catalyst Candidates in Library

a) Flow/Semi-Continuous/Batch (Non-Flow)

b) Liquid/Gas Phase Reactants

6) Optionally, Characterization of Screened Catalysts

7) Optionally, Catalyst Regeneration

8) Optionally, Screening (Reaction-Based) of Regenerated Catalyst

9) Optionally, Data Processing

10) Data Analysis—Performance Evaluation

11) Repeat One or More of Steps (1)-(10) (optionally, with automated resynthesis)

Preferably, all steps are optimized with respect to throughput, in order to eliminate unnecessary bottlenecks in the overall work flow.

Various aspects of the present invention relate to one or more different steps of the aforementioned generalized methodology. Some aspects of the invention relate to individual steps, to a combination of steps, to a particular ordering of the steps, and/or to the methodology as a whole. Note, additionally, that the various inventive aspects can be combined in any and all possible permutations, for purposes of defining the present invention. All terms used herein should be interpreted as having their ordinary meaning in the art, except and to the extent that they are further defined herein.

The following patent applications are related to the present application, particularly as applied in connection with heterogeneous catalysis research, and are specfically incorporated by reference for all purposes, including general background, methodologies, apparatus, and exemplary applications: U.S. Ser. No. 09/156,827 filed Sep. 18, 1998 by Giaquinta et al.; U.S. Ser. No. 09/518,794 filed Mar. 3, 2000 by Bergh et al.; U.S. Ser. No. 09/093,870 filed Jun. 9, 1998 by Guan et al; U.S. Ser. No. 60/185,566 filed Mar. 7, 2000 by Bergh et al; U.S. Ser. No. 09/801,390 filed Mar. 7, 2001 by Bergh et al.; U.S. Ser. No. 09/801,389 filed Mar. 7, 2001 by Bergh et al.; U.S. Ser. No. 09/285,363 filed Apr. 2, 1999 by Petro et al.; U.S. Ser. No. 09/174,856 filed Oct. 19, 1998 by Lacy et al.; and U.S. Ser. No. 09/516,669 filed Mar. 1, 2000 by Lugmair et al.

Candidate Materials/Libraries of Candidate Materials

Each of the candidate materials being screened in a combinatorial catalysis research program for a capability to enhance a chemical process of interest can be an element, a compound or a composition comprising a plurality of elements and/or compounds. The candidate material can be in a gaseous, liquid or solid phase. Solid-phase candidate materials are preferred for some applications, including generally for gas-phase heterogeneous catalysis reactions. The particular elements, compounds or compositions to be included in a library of candidate materials will depend upon the particulars of the chemical process being investigated, and upon the experimental design strategy (discussed generally above, with additional various alternatives discussed below). In general, however, the particular chemical process being investigated is not critical, and can include chemical reactions of all types.

The chemical process is preferably a chemical reaction, which for purposes hereof, means a process in which at least one covalent bond of a molecule or compound is formed or broken. As such, immunoreactions in which immunoaffinity is based solely on hydrogen bonding or other forces—while chemical processes—are not considered to be chemical reactions. In general, the candidate materials of this invention catalyze reactions that include activation of, breaking and/or formation of H—Si, H—H, H—N, H—O, H—P, H—S, C—H, C—C, C═C, C≡C, C-halogen, C—N, C—O, C—S, C—P, C—B and C—Si bonds among others. Exemplary chemical reactions for which reaction-enhancing materials may be identified according to the present invention include, without limitation, oxidation, reduction, hydrogenation, dehydrogenation (including transfer hydrogenation), hydration, dehydration, hydrosilylation, hydrocyanation, hydroformylation (including reductive hydroformylation), carbonylation, hydrocarbonylation, amidocarbonylation, hydrocarboxylation, hydroesterification, hydroamination, hetero-cross-coupling reaction, isomerization (including carbon-carbon double bond isomerization), dimerization, trimerization, polymerization, co-oligomerization (e.g. CO/alkene, CO/alkyne), co-polymerization (e.g. CO/alkene, CO/alkyne), insertion reaction, aziridation, metathesis (including olefin metathesis), carbon-hydrogen activation, cross coupling, Friedel-Crafts acylation and alkylation, Diels-Alder reactions, C—C coupling, Heck reactions, arylations, Fries rearrangement, vinylation, acetoxylation, aldol-type condensations, aminations, reductive aminations, epoxidations, hydrodechlorinations, hydrodesulfurations and Fischer-Tropsch reactions, fuel reforming reactions, water-gas-shift reactions, catalytic cracking reactions, methanol to olefin reactions, asymmetric versions of any of the aforementioned reactions, and combinations of any of the aforementioned reactions in a complex reaction sequence of consecutive reactions. For chemical reactions, the candidate materials can be generally classified as those materials which are chemically altered or consumed during the course of the reaction (e.g., co-reactant materials, cataloreactants) and those materials which are not chemically altered or consumed during the course of the reaction (e.g., catalysts, selective blocking moieties). In preferred applications, the candidate materials being evaluated in the parallel multi-variable optimization reactor are catalysts. As used herein, the term catalyst is intended to include a material that enhances the reaction rate of a chemical reaction of interest or that allows a chemical reaction of interest to proceed where such reaction would not substantially proceed in the absence of the catalyst.

The candidate materials preferably comprise elements or compounds selected from the group consisting of inorganic materials, metal-ligands and non-biological organic materials. In some applications, the candidate materials will consist essentially of inorganic materials, consist essentially of metal-ligand materials, or consist essentially of non-biological organic materials. Moreover, in some applications, the candidate materials will be compositions comprising mixtures of inorganic materials, metal-ligand materials, and/or non-biological organic materials in the various possible combinations.

Inorganic materials include elements (including carbon in its atomic or molecular forms), compounds that do not include covalent carbon-carbon bonds (but which could include carbon covalently bonded to other elements, e.g., CO₂), and compositions including elements and/or such compounds. Inorganic candidate materials that could be investigated according to the approaches described herein include, for example: noble metals such as Au, Ag, Pt, Ru, Rh, Pd, Ag, Os and Ir; transition metals such as Ti, V, Cr, Mn, Fe, Co, Ni, Cu, Zn, Y, Zr, Nb, Mo, Ta, W and Re; rare-earth metals such as La, Ce, Pr, Nd, Sm, Eu, Tb, Th and U; alloys of noble metals, transition metals and/or rare-earth metals; metal oxides such as CuO, NiO and Co₃O₄; noble-metal-doped metal oxides such as noble-metal-doped CuO, NiO and Co₃O₄; multi-metal oxides such as binary oxides of Cu—Cr, Cu—Mn, Cr—Mn, Ni—Cr, Ni—Mn, Ni—Cu, Ni—Mo, Cu—Mo, Ni—Co, Co—Mo, Ni—Fe, Fe—Mo, Cu—Fe, Mn—Ag, Mn—Sn, Ag—Sn, Cu—Ag, Cu—V, Ag—V, Cu—V, Ni—V, Bi—Mo, Bi—V, Mo—V, V—Zr, V—Ti, Zr—Ti, V—Nb, Nb—Mo, V—P, P—Mo, Ni—P, P—Cu, Co—P, Co—Fe, P—Fe, Mg—V, Mg—Sn, V—Sn, K—Ti, K—Bi, Ti—Bi, Cr—Sb, Cr—V, Sb—V, Bi—Mo, Bi—Nb, K—Cr, K—Al, Al—Cr, Zn—Cu, Zn—Al, Cu—Al, La—Cr, La—Zr, Cr—Zr, La—Mo, Mo—Zr, La—W, W—Zr, Mo—W, W—V, Cu—W, Bi—W, Fe—Sb, Fe—V and Ni—Ta, Ni—Nb and Ta—Nb, and such as ternary oxides of Cu—Cr—Mn, Ni—Cr—Mn, Ni—Cu—Mo, Ni—Co—Mo, Ni—Fe—Mo, Cu—Fe—Mo, Mn—Ag—Sn, Cu—Ag—V, Cu—Ni—V, Bi—Mo—V, V—Zr—Ti, V—Nb—Mo, V—P—Mo, Ni—P—Cu, Co—P—Fe, Mg—V—Sn, K—Ti—Bi, Cr—Sb—V, Bi—Mo—Nb, K—Cr—Al, Zn—Cu—Al, La—Cr—Zr, La—Mo—Zr, La—W—Zr, Mo—W—V, Cu—Mo—W, Bi—Mo—W, Bi—V—W, Fe—Sb—V and Ni—Ta—Nb; metal carbides such as PdC, molybdenum carbide and tungsten carbide.; metal sulfates, metal sulfides, metal chlorides, metal acetates, polyoxometallates (POM); metal phosphates such as vanadylpyrophosphates (VPO); Bronstead acids such as HF; Lewis Acids such as AlC1₃; and mixtures of any of the aforementioned inorganic materials, among others. Exemplary inorganic material libraries could include, for example, a triangular-shaped array of ternary metal oxides (e.g. such as oxides of the ternary metal partners described above) with single metal oxide compounds at each corners, binary metal oxide compositions along each of the sides with varying ratios of constituents, and ternary metal oxide compositions in the interior regions of the triangular array with varying ratios of constituents. Libraries of inorganic candidate materials can be prepared, for example, according to the methods disclosed in U.S. Pat. No. 5,776,359 to Schultz et al.

Metal-ligands comprise a central metal atom or ion surrounded by, associated with and/or bonded to other atoms, ions, molecules or compounds—collectively referred to as “ligands”—typically through a carbon (to form, e.g., an organometallic), nitrogen, phosphorous, sulfur or oxygen atom and/or one or more linker moieties. The one or more ligands typically bind to one or more metal center and/or remain associated therewith, and by such association, modify the shape, electronic and/or chemical properties of the active metal center(s) of the metal-ligand complex. The ligands can be organic (e.g., η¹− aryl, alkenyl, alkynyl, cyclopentadienyl, CO, alkylidene, carbene) or inorganic (e.g., Br⁻, Cl⁻, OH⁻, NO²⁻, etc.), and can be charged or neutral. The ligand can be an ancilliary ligand, which remains associated with the metal center(s) as an integral constituent of the catalyst or compound, or can be a leaving group ligand, which may be replaced with an ancillary ligand or an activator component. Exemplary metals/metal ions include ions derived from, for example, simple salts (e.g., AlCl₃, NiCl₂, etc.), complex or mixed salts comprising both organic and inorganic ligands (e.g., [(η5-C₅Me₅)IrCl₂]₂, etc.) and metal complexes (e.g., Gd(NTA)₂, CuEDTA, etc.), and can generally include, for example, main group metal ions, transition metal ions, lanthanide ions, etc.

Libraries of metal-ligand candidate materials can be prepared, for example, according to the methods disclosed in PCT Patent Application WO 98/03521 of Weinberg et al. Briefly, a desired ligand can be combined with a metal atom, ion, compound or other metal precursor compound. In many applications, the ligands will be combined with such a metal compound or precursor and the product of such combination is not determined, if a product forms. For example, the ligand may be added to a reaction vessel at the same time as the metal or metal precursor compound along with the reactants. The metal precursor compounds may be characterized by the general formula M(L)ₙ (also referred to as MLₙ or M-Lₙ) where M is a metal and can include metals selected from the group consisting of Groups 5, 6, 7, 8, 9 and 10 of the Periodic Table of Elements. In some embodiments, M can be selected from the group consisting of Ni, Pd, Fe, Pt, Ru, Rh, Co and Ir. L is a ligand and can be selected from the group consisting of halide, alkyl, substituted alkyl, cycloalkyl, substituted cycloalkyl, heterocycloalkyl, substituted heterocycloalkyl, aryl, substituted aryl, heteroaryl, substituted heteroaryl, alkoxy, aryloxy, hydroxy, boryl, silyl, hydrido, thio, seleno, phosphino, amino, and combinations thereof, among others. When L is a charged ligand, L can be selected from the group consisting of hydrogen, halogens, alkyl, substituted alkyl, cycloalkyl, substituted cycloalkyl, heteroalkyl, heterocycloalkyl, substituted heterocycloalkyl, aryl, substituted aryl, heteroaryl, substituted heteroaryl, alkoxy, aryloxy, silyl, boryl, phosphino, amino, thio, seleno, and combinations thereof. When L is a neutral ligand, L can be selected from the group consisting of carbon monoxide, isocyanide, nitrous oxide, PA₃, NA₃, OA₂, SA₂, SeA₂, and combinations thereof, wherein each A is independently selected from a group consisting of alkyl, substituted alkyl, heteroalkyl, cycloalkyl, substituted cycloalkyl, heterocycloalkyl, substituted heterocycloalkyl, aryl, substituted aryl, heteroaryl, substituted heteroaryl, alkoxy, aryloxy, silyl, and amino. Specific examples of suitable metal precursor compounds include Pd(dba)₂ (dba=dibenzylydieneacteone), Pd₂(dba)₃, Pd(OAc)₂ (Ac=acetate), PdCl₂, Pd(TFA)₂, (TFA=trifluoroacetate), (CH₃CN)₂PdCl₂, and the like. In this context, the ligand to metal precursor compound ratio is in the range of about 0.01:1 to about 100:1, more preferably in the range of about 0.5:1 to about 20:1. The metal atom, ion or metal precursor may be supported or not. Supports may be organic or inorganic. Similar to the ligands, the support may be an L. In other embodiments, the support will not form part of the metal precursor and suitable supports include silicas, aluminas, zeolites, polyethyleneglycols, polystyrenes, polyesters, polyamides, peptides and the like. Specific examples of Pd supported metals include Pd/C, Pd/SiO₂, Pd/CaCO₃, Pd/BaCO₃, Pd/aluminate, Pd/aluminum oxide, Pd/polystyrene, although any of the metals listed above could replace Pd in this list, e.g., Ni/C, etc. In other applications, the ligand will be mixed with a suitable metal precursor compound prior to or simultaneous with allowing the mixture to be contacted to the reactants. When the ligand is mixed with the metal precursor compound, a metal-ligand complex may be formed, which may be employed as a candidate material.

Non-biological organic materials include organic materials other than biological materials. Organic materials are considered to include compounds having covalent carbon-carbon bonds. Biological materials are considered to include nucleic acid polymers (e.g., DNA, RNA) amino acid polymers (e.g., enzymes) and small organic compounds (e.g., steroids, hormones) where the small organic compounds have biological activity, especially biological activity for humans or commercially significant animals such as pets and livestock, and where the small organic compounds are used primarily for therapeutic or diagnostic purposes. While biological materials are of immense commercial interest with respect to pharmaceutical and biotechnological applications, a large number of commercially significant applications involve chemical processes that are enhanced by other than biological materials. Moreover, while fundamental screening approaches for many pharmaceutical and biological activities are known or readily adapted from known approaches, screening approaches for non-biological materials have not heretofore been widely investigated and reported. Although the candidate materials being screened are preferably not, themselves, biological organic materials, the candidate materials of the invention (e.g., inorganic materials) can be employed to enhance reactions directed to producing a biological organic material as the product of a chemical reaction (e.g., materials that enhance chemical-based, non-enyzmatic DNA synthesis, or materials that enhance a synthetic, non-enyzmatic route to a particular hormone or steroid).

In preferred applications, the candidate materials are catalysts being screened for catalytic activity and/or for catalytic selectivity for a chemical reaction of interest. The candidate catalysts can be homogeneous catalysts or heterogeneous catalysts. For homogeneous catalysis, the candidate materials are preferably solids or liquids which are soluble or miscible in the reaction medium under the reaction conditions, but can also include gasses. For heterogeneous catalysis, the candidate materials are preferably solids. In general, homogeneous candidate catalyst materials and heterogeneous candidate catalyst materials can include organic, inorganic and metal-ligand catalysts such as are described above. Exemplary reactions for which a homogeneous catalyst may be investigated pursuant to the present invention, as well as known homogeneous catalysts for such reactions are shown in Table 1 A. Exemplary reactions for which a heterogeneous catalyst may be investigated pursuant to the present invention, as well as known heterogeneous catalysts for such reactions are shown in Table 1B. The library of candidate catalysts being screened can be variations in the structure or composition of known catalysts or can be structurally unrelated thereto.

TABLE 1A
Exemplary Homogeneous Catalytic Reactions

| Reaction Class | Known Catalyst |
|---|---|
| assymetric C-C double | Ru—, Rh— ligand (e.g., phosphine) |
| bond isomerization | |
| Suzuki biaryl cross-coupling | Pd-ligand (e.g., phosphine) |
| hydroformylation | Co—, Rh— ligand (e.g., phosphine, |
| | phosphite) |
| hydrocarboxylation | Mo—, Pd—, Rh—, Co—, ligand (e.g., |
| | phosphine) |
| Heck reaction | Pd-ligand (e.g., phosphine) |
| hydrocyanation | Ni-ligand (e.g., phosphite) |
| assymetric hydrogenation | Ru—, Rh— ligand (e.g., phosphine) |
| Friedel-Crafts reaction | HF, AlCl₃ |
| olefin polymerization | Zr—, Ti—, Hf— ligand (e.g. cyclo- |
| | pentadiene) Ni—, Pd— ligand (e.g., |
| | bis-imine) |
| olefin metathesis | Ru—, Mo— ligand (e.g., N—, P— based) |
| methanol carbonylation | Ir, Rh with halides (e.g., MeI, HI) |
| epoxide ring opening | Cu-ligand (e.g., alkoxide, amide, amine) |

TABLE 1B
Exemplary Heterogeneous Catalytic Reactions

| Reactant(s) | Product | Known Catalyst |
|---|---|---|
| ethylene + acetic | vinyl acetate | Pd—Au |
| acid | | |
| ethylene glycol | glyoxal | Cu |
| ethylene | ethyleneoxide | Ag |
| methanol | formaldehyde | Ag |
| butene dimerization | octene | Ni |
| HCl | Cl2 | Cu—Fe—Cl, Cu—Cr—O |
| propylene | acrolein | Bi—Mo—O |
| acrolein | acrylic acid | Mo—V—O(+Cu—Mo—O+ |
| | | MnO + W-O) |
| methacrolein | methacrylic acid | POM |
| o-xylene | Phthalic anhydride | V/TiO2 |
| butane | maleic anhydride | VPO |
| toluene | benzonitrile | V—Sb—O, Fe—Sb—O |
| toluene | benzaldehyde | Co acetate |
| toluene | benzoic acid | Co acetate |
| ethylbenzene | styrene (non-ODH) | K—Fe—O |
| ethylbenzene | styrene (ODH) | K—Bi—O/TiO2 |
| propane | propylene | K—Cr—O/Al2O3 |
| vinyl cyclohexene | styrene | Cu/zeolite |
| cyclohexanol | cyclohexanone | Cu/SiO2 |
| cyclohexene | benzene | NM/support |
| cyclohexylamine | aniline | NM/support |
| side chain aromatics | aromatic acids | Co—Mn—Zr-acetates |
| ethylene | acetaldehyde | Pd—Cu, Pd—Au |
| acetaldehyde | acetic acid | Mn-acetate |
| propylene | propylene oxide | Ti/silicalite, Au/TiO2 |
| propylene | acrylic acid | |
| propylene | acrylonitrile | |
| butadiene | vinyl oxirane | Ag |
| nitrobenzene | aniline | Cu/SiO2, supported NM |
| beta-picoline | nicotinic acid | V—Mo/Ti—Zr—O |
| maleic anhydride | gamma- | Cu—Zn—O, Cu—Cr—O |
| | butyrolactone, | |
| | tetrahydrofurane | |
| propane | acrylic acid | V—Mo—Nb—O—Te |
| propane | acryl nitrile | V—Sb—O |
| benzene | phenol | Fe—Ga/zeolite |
| syngas | methanol | Cu—Zn/Al2O3 |
| syngas | methane | Ni |
| syngas | fuel hydrocarbons | Fe, Co |
| H2 + N2 | ammonia | Fe |
| CH4 + H2O | H2 + CO | Ni |
| DeNOx | | V/TiO2 |
| CH3OH | olefins | SAPO34 |
| acetylene | ethylene | PdAg, PdAu |

A library of candidate materials comprises four or more, and preferably a higher number of diverse candidate materials. The library of materials is preferably arranged in an array, preferably comprising the diverse materials in spatially determinative regions (e.g., within different reaction vessels or modules comprising reaction vessels), and most preferably in spatially determinative and distinct regions (e.g., regions defined in one or more substrates). Modules comprising reaction vessels within a single reaction apparatus can each comprise a single substrate, and/or can collectively be considered as part of a larger substrate. The materials are preferably catalysis materials, such as candidate catalysts, or precursors thereof (including catalyst supports).

A library of catalysis materials, and/or a synthesis or screening protocol for such a library, can be characterized as (and in general, should be considered generic to, unless specifically recited otherwise) a primary screen, a secondary screen, a ternary screen, a quaternary screen, and/or a higher-order screen. The library and/or synthesis or screening protocols can likewise be characterized as (and in general, should be considered generic to) an initial library/screen directed toward initial identification of hits or leads, or a related, subsequent focus library/screen. See, for example, as previously described in U.S. Ser. No. 09/518,794 filed Mar. 3, 2000 by Bergh et al. The number of catalysis materials in the library or array is preferably four or more, preferably six or more, more preferably eight or more, sixteen or more, twenty-four or more, forty-eight or more, ninety-six or more, two-hundred or more, four hundred or more, one thousand or more, four thousand or more, ten thousand or more, or in some embodiments, 96*N, where N ranges from 1 to about 20, and preferably from 1 to about 5.

In preferred embodiments, the plurality of catalysts or catalyst precursors (e.g., including catalyst supports) of the library are different from each other with respect to composition and/or concentration. The compositional space of the library can typically comprise four or more diverse compositions having one or more common elements at various concentrations or stoichiometries (a unitary library), preferably two or more common elements at various concentrations or stoichiometries (a binary library) more preferably three or more common elements at various concentrations or stoichiometries (a ternary library), or a higher-order library (e.g., a quaternary library). See U.S. Pat. No. 5,985,356 to Schultz et al., and U.S. patent application Ser. No. 09/156,827 filed Sep. 18, 1998 by Giaquinta et al. In a ternary library comprising elements A, B, and C, for example, each of A, B and C can range from 0% to 100% within the ternary library at various stoichiometric increments (e.g., at 10% increments). The library can also include one or more standard compositions present at a plurality of test regions (e.g., reaction vessels or reaction sites) of an array. In some embodiments, a standard composition is preferably present at three or more test regions, four or more test regions, six or more test regions, or eight or more test regions.

The library of catalysis materials can also be developed and differentiated with respect to process conditions. Generally, process conditions refers, inclusively, to (i.e., is intended as being generic to) synthesis protocols (e.g., precipitation, impregnation, spray drying, etc.), synthesis conditions within a particular synthesis protocol, pretreatment protocols (e.g., physical pretreatments such as heating or calcining, mechanical pretreatments such as grinding, mixing, pressing, crushing, sieving, and/or chemical pretreatments such as reduction (e.g., by H2, C2H4, etc.), activation (e.g., by C2H4), partial oxidation, etc., pretreatment conditions within a particular pretreatment protocol, reaction conditions (e.g., selected from the group consisting of temperature, pressure, space velocity and contact time, and especially selected from the group consisting of space velocity and contact time), regeneration conditions (e.g., post-reaction treatments prior to reuse), and any other catalytically significant process variables prior to, during, or subsequent to catalytic (reaction-based) screening of the candidate catalyst material for a particular reaction (or reactions) of interest.

Variations in process conditions can, in general, be simultaneous (i.e., parallel variation in conditions), serial, or semi-parallel (i.e., serial with respect to a parallel subset), but are preferably parallel variations in process condition. Reaction conditions for synthesis can be varied within elements of an array, or between different arrays. See for example, U.S. Pat. No. 6,004,617 to Schultz et al. Reaction conditions during screening can also be varied between different arrays and/or within elements of an array, as discussed above, and as further detailed for simultaneously variation of reaction conditions using a multi-variable optimization reactor (MVO) such as that described in U.S. Ser. No. 60/185,566 filed Mar. 7, 2000 by Bergh et al., U.S. Ser. No. 09/801,390 filed Mar. 7, 2001 by Bergh et al., and U.S. Ser. No. 09/801,389 filed Mar. 7, 2001 by Bergh et al. Catalytic performance can be characterized by any suitable performance-indicating parameter. Conversion and selectivity for a particular reaction of interest are particularly preferred. See, for example, U.S. Ser. No. 09/518,794 filed Mar. 3, 2000 by Bergh et al.; see also U.S. Ser. No. 09/093,870 filed Jun. 9, 1998 by Guan et al.

The libraries can include one or more representative compositions. A representative composition is a composition that is compositionally representative of a compositional space to be evaluated in one or more subsequent screening experiments (e.g., one or more parallel screening evaluations). Such compositional space can typically be defined by a library comprising a plurality of diverse compositions having one or more common elements at various concentrations (a unitary library), preferably two or more common elements at various concentrations (a binary library) more preferably three or more common elements at various concentrations (a ternary library), or a higher-order library (e.g., a quaternary library). See U.S. Pat. No. 5,985,356 to Schultz et al., and U.S. patent application Ser. No. 09/156,827 filed Sep. 18, 1998 by Giaquinta et al. The representative composition is a composition included within the compositional confines of the space to be evaluated. A preferred representative composition of a ternary library comprising elements A, B, and C, each ranging from 0% to 100% within the ternary library, can comprise, for example, ⅓A, ⅓B and ⅓C.

The libraries can also include one or more standard compositions. A standard composition is a composition present at a plurality of test regions (e.g., reaction vessels or reaction sites) of an array, and in some embodiments, is preferably present at three or more test regions, four or more test regions, six or more test regions, or eight or more test regions.

The libraries can, generally, comprise candidate catalysis materials that include one or more catalyst platforms. A catalyst platform, as used herein, is intended to refer to a plurality, and preferably higher numbers (as described herein and in the related applications/patents), that have common or related chemical (molecular) composition or structure. Exemplary catalyst platforms include supported or bulk mixed metal oxides (MMO's), noble-metal/base metal (NM/BM) or oxides thereof, polyoxometallates (POM's), zeolites and other related, microporous and mesoporous materials, among others. The members of a catalyst platform can be presynthesized, and available in library format as source materials, which can in application, be daughtered for use in synthesis protocols to prepare of the arrays of the present inventions.

As noted, the set of parallel experiments to performed simultaneously, preferably in an integral parallel reactor, involve controlled variations in process conditions, such as reaction conditions. More generally, process conditions refer, inclusively, to (i.e., is intended as being generic to) synthesis protocols (e.g., precipitation, impregnation, spray drying, etc.), synthesis conditions within a particular synthesis protocol, pretreatment protocols (e.g., physical pretreatments such as heating or calcining, mechanical pretreatments such as compaction, grinding, sieving, and/or chemical pretreatments such as reduction (e.g., by H2, C2H4, etc.), activation (e.g., by C2H4), partial oxidation, etc., pretreatment conditions within a particular pretreatment protocol, reaction conditions (e.g., selected from the group consisting of temperature, pressure, space velocity and contact time, and especially selected from the group consisting of space velocity and contact time), regeneration conditions (e.g., post-reaction treatments prior to reuse), and any other catalytically significant process variables prior to, during, or subsequent to catalytic (reaction-based) screening of the candidate catalyst material for a particular reaction (or reactions) of interest.

Preliminary Evaluation of Process Conditions, With Simultaneous or Subsequent Evaluation of Compositional Space)

Generally, evaluation of process conditions prior to or concurrently with a study of compositional space offers substantial efficiencies over prior art approaches of exploring compositional space first, through one or more screening experiments, to identify hits and/or leads, and then subsequently optimizing process conditions for the identified hits and/or leads.

Prescreening of Representative Composition at Various Process Conditions

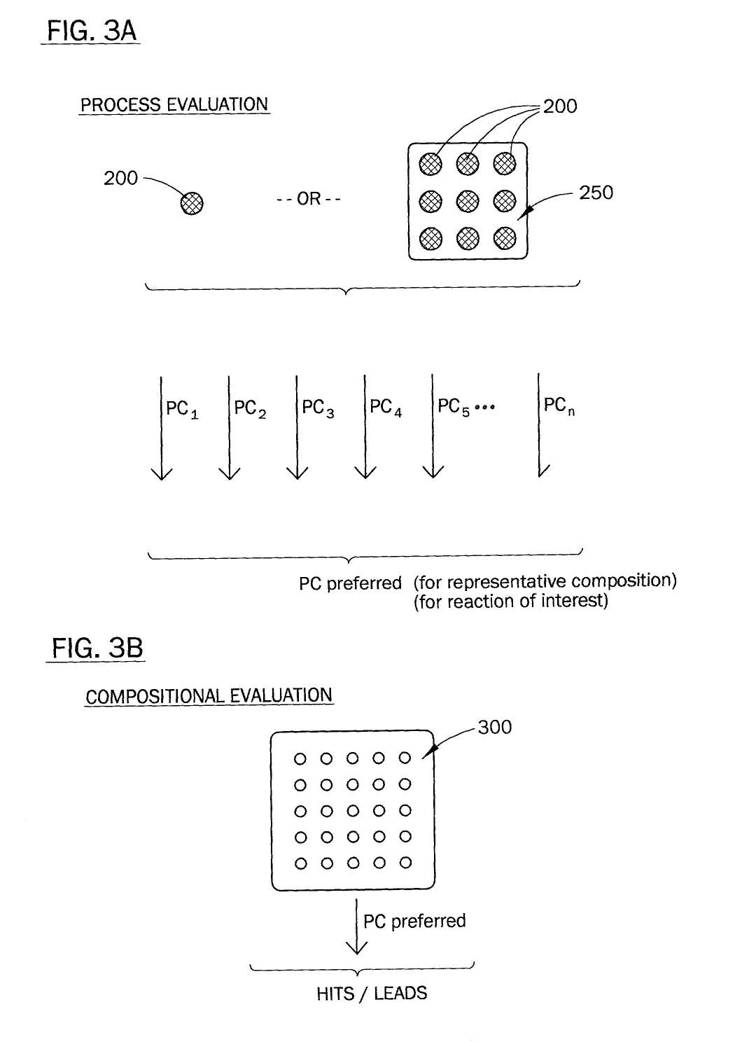

In one embodiment, with reference to FIG. 3A, a representative composition 200 or a library comprising an array of representative compositions 200 (e.g., on a common support 250) can be evaluated for catalytic performance at a plurality, preferably at least three or more, and most preferably at least four or more, five or more, six or more, seven or more, or eight or more different process conditions (PC1, PC2, PC3, PC4, PC5, . . . PCn) (e.g., different reaction conditions, different synthesis protocols, etc.). One or more of the evaluated process conditions are identified as being preferable relative to other evaluated process. An array 300 comprising a library of diverse candidate materials that define the compositional space to be explored is subsequently screened at, substantially at, or between one or more of the preferred process conditions Ppreferred identified in the earlier screening experiment(s). Further compositional and or process optimization can then be effected with additional libraries/screens, as desired.

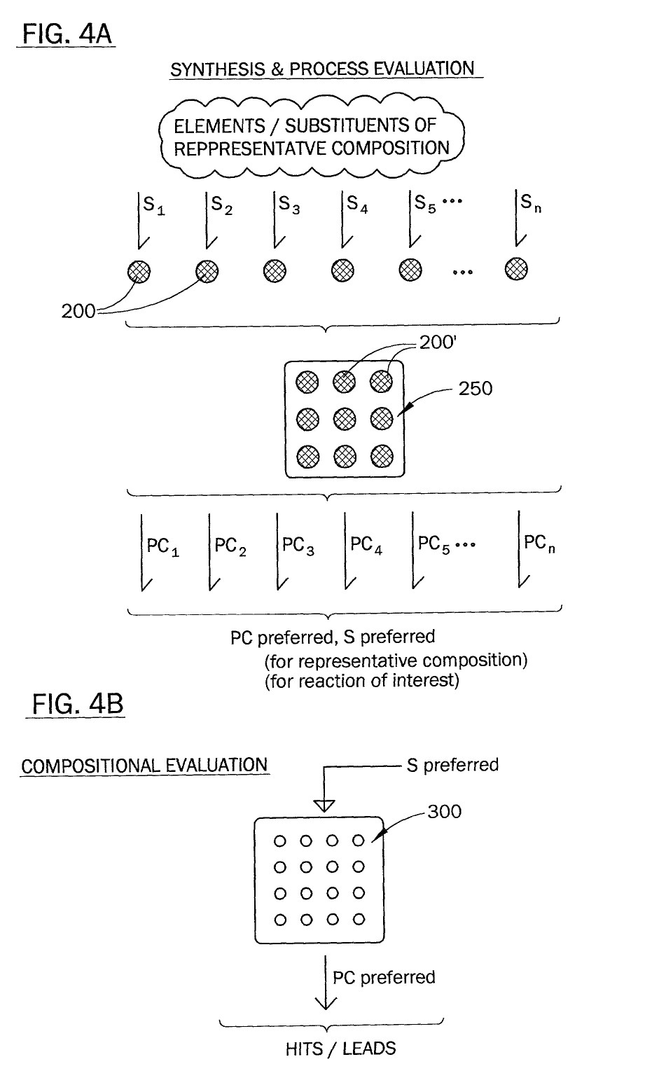

In another embodiment, constituting a variation of the aforementioned embodiment, the representative composition is, with reference to FIG. 4A, evaluated in one set of experiments at various reaction conditions (PC1, PC2, PC3, PC4 . . . PCn), and in another set of experiments with respect to various synthesis protocols (or alternatively, various synthesis conditions for a particular synthesis protocol) or pretreatment protocols (generally indicated in FIG. 4A as S1, S2, S3, S4, . . . Sn), in each case, substantially as described in the immediately preceding embodiment. One or more preferred reaction conditions (e.g., Ppreferred), and one or more preferred synthesis protocols (e.g., Spreferred) are determined. A third set of reaction screening experiments—involving a compositionally diverse array 300 synthesized using the preferred synthesis protocols (Spreferred) is then screened in a reaction-based screen to evaluate compositional diversity (represented schematically in FIG. 4B) under preferred reaction conditions, Ppreferred.

Simultaneous Evaluation of Process Conditions and Initial Compositions, with Subsequent Evaluation of one or more Compositional Focus Libraries

In a variation of the aforementioned approach, an array comprising a representative composition (e.g., as a standard composition) at one or at a plurality of test regions (e.g., reaction vessels) (e.g., preferably three or more, and most preferably four or more different, spatially determinative regions of a substrate), and preferably a compositionally-diverse array, is evaluated for catalytic performance at a plurality, preferably at least three or more, and most preferably at least four or more, five or more, six or more, seven or more, or eight or more different process conditions (e.g., different reaction conditions, different synthesis protocols, etc.). One or more of the evaluated process conditions are identified as being preferable relative to other evaluated process. Additionally, one or more of the diverse compositions are identified as being preferable, with respect to catalytic performance, relative to other evaluated compositions. An array comprising a focus library based on the identified preferred candidate catalysts that define a different (e.g., focused) compositional space is subsequently screened at, substantially at, or between one or more of the preferred process conditions identified in the earlier screening experiment(s). Further compositional and or process optimization can then be effected with additional libraries/screens, as desired

Simultaneous Evaluation of Process Conditions and Array of Initial Compositions, where Initial Compositions Comprise Two or More Different Catalyst Platforms, With Subsequent Evaluation of one or more Libraries of a Preferred Catalyst Platform

Evaluation of process conditions using an array comprising broadly diverse, compositional variations is also advantageous. In a preferred embodiment, an array comprising at least two or more, or in some cases, at least three or more, or at least four or more different catalyst platforms (e.g., mixed-metal oxides (MMO's), zeolites and other related mesoporous or microporous materials, noble-metal/base metals (NM/BM), polyoxometallates (POM's), and others, as supported catalysts or unsupported (bulk) catalysts) is evaluated first with respect to process optimization. Specifically, the multi-platform array is evaluated for catalytic performance at a plurality, preferably at least three or more, and most preferably at least four or more, five or more, six or more, seven or more, or eight or more different process conditions (e.g., different reaction conditions, different synthesis protocols, etc.). One or more of the evaluated process conditions are identified as being preferable relative to other evaluated process. Additionally, one or more of the different catalyst platforms are identified as being preferably relative to other evaluated catalyst platforms. An array comprising a library of candidate catalysts of the identified catalyst platform is subsequently screened at, substantially at, or between one or more of the preferred process conditions identified in the earlier screening experiment(s). Further compositional and or process optimization can then be effected with additional libraries/screens, as desired.

Simultaneous Evaluation of Process Conditions/Initial Compositions with Predictive Extrapolation

Evaluation of process conditions with predictive extrapolation is likewise advantageous with respect to efficiency. In a preferred embodiment, with reference to FIG. 5A, an array 300 comprising compositionally varying candidate catalysts (e.g., varied as described in above with respect to composition), together with at least one “standard” composition (preferably from the same library family being evaluated) that is repeated at a plurality, preferably three or more, and most preferably at least four or more different test regions (e.g., reaction vessels or wells of a substrate—or in different modules comprising reaction vessels) is evaluated for catalytic performance under a plurality of process conditions, and preferably under at least three or more, and most preferably at least four or more, five or more, six or more, seven or more, or eight or more different process conditions (e.g., different reaction conditions, different synthesis protocols, etc.), with different process conditions for each of the plurality of standard compositions in the array.

The catalytic performance of the standard composition under different process conditions provides a basis for statistical analysis of the results, which can be used to extrapolate the results for the varied compositions to the different process conditions. Specifically, with reference to FIG. 5B, for example, a master curve of determined performance characteristics (e.g., selectivity, S, versus conversion, C) can be plotted for the standard composition at each of the different process conditions. The master curve for the standard composition, together with catalyst performance data for one or more of the evaluated compositions of the original array at one or more of the evaluated process conditions (preferably at two or more, three or more, or four or more thereof), can then be used to predict, using mathematical models (e.g., computer-implemented algorithms) a master curve for each of the other compositions, thereby essentially defining the entire performance surface function for the screened compositional and process-varying space.

The predictive value, while certainly improving overall screening efficiency (e.g., defined as an experiments per hour basis), relies on certain assumptions, including the assumption of a smooth (relatively smooth) performance surface function for the reaction of interest for the parameter space (e.g., compositional, process condition) being investigated, and on the applicability of the mathematical model to define the relationship between the empirically-determined master curve for the standard composition, and the predicted master curve for the other screened compositions. Somewhat surprisingly, research to date involving heterogeneous catalysis of several hundreds of thousands of experiments (combinatorial, high-throughput experiments) for several different reaction systems, has confirmed the validity of a smooth performance surface function.

Mathematical models can be determined from literature, if possible, and/or postulated and evaluated specifically for the reaction of interest. Predictive value can be improved empirically, for example, using a larger number of empirically-derived data points for the compositions being evaluated, as shown in FIG. 5C (with four data points), although, as a tradeoff, there is a reduction in overall efficiency.

According to one exemplary embodiment, the above-described protocols involving simultaneous evaluation of process conditions with predictive extrapolation can be effected in a parallel flow reactor, such as a 48-channel fixed bed reactor described in the related patent applications and in U.S. Pat. No. 6,149,882 to Guan et al., and configured as eight modules, each comprising six reaction vessels. Several approaches are described herein for improving the efficiency of initial process screening in the multi-channel parallel reactor.

FIG. 5A shows a schematic representation of a 48-channel parallel flow reactor comprising eight modules (represented as rows) having independent reaction condition control, where each of the eight modules comprises six reaction vessels (represented as rectangles, eight per row, in six columns). In one embodiment, at least one reactor vial in each row can comprise a standard catalyst or catalyst precursor. As discussed above, the performance of this standard catalyst towards different operating conditions provides a basis for statistical analysis of the results, and extrapolation of results for the non-standard catalysts or precursors. The reliability of the prediction can be improved, in another embodiment, by using more than one standard at two or more of the reaction conditions, for example, as shown in FIG. 5A.

The usefulness of this strategy screening strategy can be demonstrated by comparing a non-predictive approach with the predictive approach of this aspect of the invention. As a basis of comparison, assume that the time required for screening and analysis of the array of catalysts is about 10 minutes per row, using three, two-channel gas chromatographs. Under such assumption, it would take about 80 minutes to run an entire library of 48 samples. An entire library of 48 samples is screened—without using the standards catalysts and without using the predictive extrapolation approach of the present invention—for example, at eight different reaction conditions. To screen the 48 catalysts under the 8 different reaction condition requires 8×48=384 data points, and a total of 10.66 hours. The throughput is 36 data points/hour. It may be noted that here we are not taking into account the problems due to possible catalyst deactivation as a function of reaction time.

If a library is employed comprising (i) a standard catalyst in one reaction vessel of each of the eight modules, as shown in FIG. 5A (shaded region in column indicating standard catalysts), as well as (ii) duplicates of each of twenty (20) candidate catalysts in the remaining 5 columns of the 6×8 array of reaction vessels. Thus the library has duplicates of 20 different catalysts and six copies of 1 standard catalyst. Each of the eight modules (rows) are screened with each module being at a different reaction condition, as noted in FIG. 5A. This screening generates 48 real data points for catalytic performance (e.g., conversion, C, and selectivity, S) for the reaction of interest. With reference to FIG. 5D, the forty-eight data points are plotted onto a master curve of selectivity versus conversion (or analyzed equivalently, without actual plotting, using automated software). The standard catalyst has eight different data points (indicated as x's in FIG. 5D) corresponding to each of the eight different reaction conditions at which the standard composition was screened. Each of the duplicates of the other twenty catalyst candidate compositions are also plotted on the master curve (shown, in FIG. 5D, only for two of such compositions, composition 1 with actual empirical data at reaction conditions PC3 and PC6, and composition 2, with actual empirical data at reaction conditions PC2 and PC5). Based on the two actual data points for each of the twenty different library members, and based on the data for the standard catalyst composition, mathematical model algorithms can then used to extrapolate and predict the performance characteristics for the twenty compositions at each of the other reaction conditions—even though they were not actually empirically screened at such conditions. As a figure of efficiency gain, note that the total information gleaned from the experiment, including both actual empirical data and statistically derived “predicted” data, includes 168 data points (48 real data points +120 extrapolated data points representing 21 different catalysts under 8 different reaction conditions). The throughput is therefore 111 data points per hour (compared to 36 data points per hour, without the predictive approach).

In another exemplary approach, in which some gain in efficiency is achieved, but the predicted data has enhanced reliability, one entire row can be loaded with the standard catalyst composition as shown in FIG. 5A. The remaining reaction vessels comprise ten different candidate catalysts, with each of the different catalysts being repeated four times (instead of duplicates, as in the previous example). Hence, as depicted in FIG. 5E, the parallel reactor screens 10 different catalysts (each at four different reaction conditions) and one standard (at eight different reaction conditions). Again, this generates only 48 real empircal data points, but the information can be extrapolated to 40 additional predicted data points (10 different catalysts at four different reaction conditions) for a total of 88 total data points. This happens in 1.33 hrs, and therefore the throughput is 66 data points per hour.

Basis for Catalyst Evaluation other than Weight

Catalysts can be supplied to reaction screening apparatus on a equal weight basis, on an equal volume basis or independently of either weight or volume—for comparison between different channels of the reactor. In view of the substantial amount of time to effect such weighing for numerous catalyst candidates, however, and the inherent potential for inaccuracies, comparison without a weight basis can be advantageous.

Catalyst Evaluation with Volumetric Basis, Rather than Weight (Mass)Basis

Catalyst libraries (e.g., with different catalyst candidates) are preferably screened in parallel, and the relative catalytic performance of the candidate catalysts is determined, preferably simultaneously. The comparative basis of the determined performance results being provided through equal volumes—rather than equal weights, necessarily—of catalysts, catalyst supports, and/or catalyst precursors. Such an approach is particularly advantageous with respect arrays comprising higher numbers of library members (e.g., ten or more, preferably twenty or more, more preferably forty or more, even more preferably ninety or more, and most preferably one-hundred and fifty or more library members) due to the relatively less tedious, ease of parallelization of volumetric dispensing protocols, as compared to weighing protocols. Moreover, arrays of candidate catalysts are preferably prepared and screened using a volumetric comparative basis, since the useful catalyst metrics and reaction parameters such as contact time, space velocity and space-time-yield, are defined with respect to catalyst volume and not weight. Hence, arrays of candidate catalysts are preferably prepared by providing substantially the same volume of a catalyst support, catalyst precusor, and/or catalyst for the various candidate catalysts of the array, most preferably without weighing the support, catalyst precursor and/or catalyst, and then screening the array to determine the relative catalytic performance.

In a preferred approach for volumetric-based arrays comprising supported catalysts or catalyst precursors, the supports (e.g., alumina, silica, etc.) comprise microspheres or other microparticles that are substantially the same size and/or otherwise substantially identical with respect to packing, such that equi-volumes is substantially equivalent to equi-weights. The particle size distribution (e.g., diameter of spheres) preferably varies by not more than about 20%, more preferably not more than about 15%, even more preferably not more than about 10%, still more preferably not more than about 5%, yet more preferably not more than about 3%, more preferably still not more than about 2% and most preferably not more than about 1%. The actual particle size employed is not narrowly critical, and can vary depending on the particular chemical reaction of interest. In some applications, however, it is preferable to employ particles with the aforementioned particle size distribution and having a size (e.g., diameter) ranging from about 1/10 to about 1/20 of the size (e.g., diameter) of the fixed-bed reactor used in the reaction screening. Typical diameters for particle size can range, for example, from about 100 um to about 2 mm, and preferably from about 200 um to about 500 um. For a 4 mm inside diameter fixed-bed reaction zone, for example, a preferred particle size ranges from about 200 urn to about 400 um.

Catalyst Evaluation Independent of Weight (Mass), and Optionally, also Volume

In another preferred approach for determining the relative performance of catalyst candidate materials, an array comprising supported or unsupported catalysts, and/or catalysts precursors can be screened under reaction conditions (e.g., temperature, pressure, space velocity and/or contact time) that are systematically varied (e.g., as generally described above) for each of the different catalyst/precursor materials to determine performance characteristics (e.g., conversion, C, and/or selectivity, S, etc.) at each of the various reaction conditions. The different process conditions can include a plurality of process conditions, and preferably at least three or more, and most preferably at least four or more, five or more, six or more, seven or more, or eight or more different process conditions (e.g., different reaction conditions, different synthesis protocols, etc.). For reaction conditions, variation in temperature and space velocity are particularly preferred. The catalysts and/or catalyst precursors can be prepared independently of (i.e., without consideration of) the mass of materials, and optionally but less preferably, also independently of volume of materials.

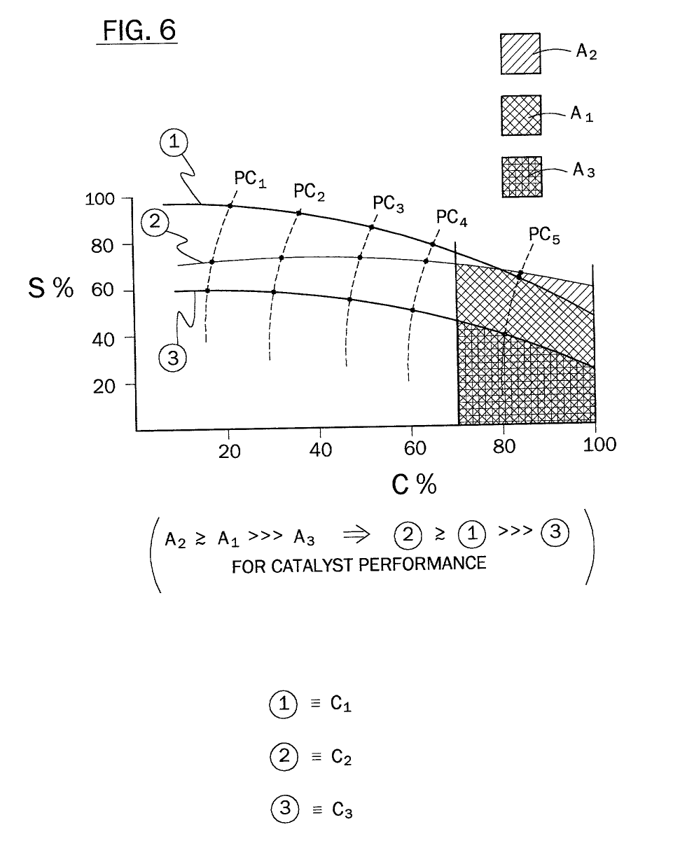

The performance characteristics at the various process conditions can then be plotted for each of the library members to obtain a master curve plot (e.g., C vs. S for each of the various library members, based on data from the different process conditions). Evaluation of the relative performance of the candidate catalysts can be effected by comparing their respective master curves. As one measure of comparison, for example, the relative areas under the respective master curves can be compared. Alternatively, a weighted area—for example, with reference to FIG. 6, considering the area encompassed by the curve above a particular target selectivity (e.g., 70%) and a particular conversion (e.g, 20%)—can be compared for each of the candidate catalysts, to determine the best-performing catalyst(s). Advantageously, knowledge of the sample weight for each particular library member is not required for comparative evaluation. That is, evaluation of catalyst performance while varying process conditions (e.g., space velocity) provides a comparative basis between the different library members that is independent of weight of candidate material, and optionally, also independent of volume of candidate material.

Selective Active-Phase Synthesis (Heterogeneous Catalysis Synthesis Kit)

In a preferred catalyst or catalyst precursor preparation approach, catalytically active or promoter phases for a reaction of interest are identified, for example, through scooping experiments (e.g., involving experimental work-ups) and/or through relevant literature. For each catalyst or catalyst precursor of a combinatorial heterogeneous catalyst library comprising four or more, and preferably higher numbers of such catalyst 1 catalyst precursor, two or more catalytically active or promoter phases (e.g. comprising a single element (i.e. a single phase), or comprising two or more elements (e.g., binary, ternary, quaternary and/or higher-order phases)) are preformed as separate components defined by distinct phases. The preformed components have an essential, substantial absence of detrimental compositional phases—phase compositions that would be detrimental to catalytic performance for the reaction of interest. The catalytically active components are subsequently combined (e.g., physically, through integral mixing of particles, such as through the parallel compaction, grinding and sieving process disclosed above) to form the catalyst candidate or catalyst precursor. Following optional pretreatment (e.g., moderate temperature calcining—such as less than about 500 C), the library of candidate catalysts can be screened in high-throughput (preferably simultaneously, in parallel) for catalytic performance for the reaction of interest.

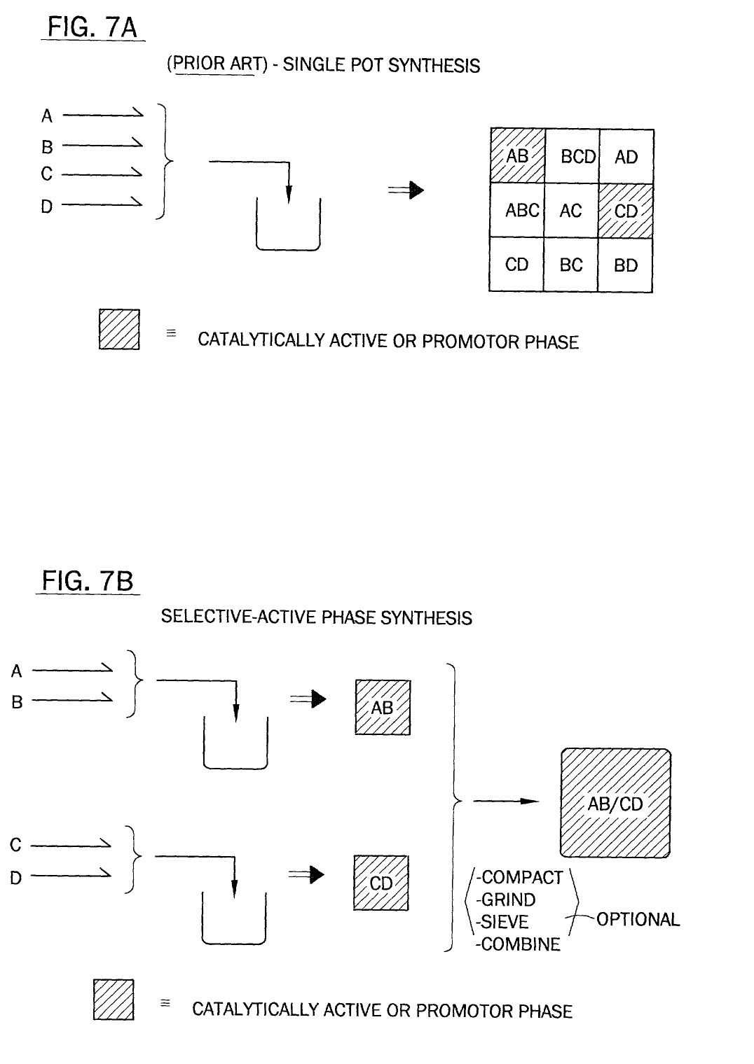

The aforementioned approach offers substantial advantages over conventional “single-spot” approaches to forming combinatorial libraries of candidate heterogeneous catalysts. Such single-pot synthesis protocols necessarily result in the formation of all thermodynamically possible phases for the elements of interest used in the synthesis—without regard to whether such formed phases are beneficial or detrimental to catalyst activity and/or promotion. In contrast, the preliminary identification of, and preforming of the catalytically active or promoting phases for a reaction of interest avoids the formation of and presence of catalytically detrimental phases in the candidate materials. With reference to FIGS. 7A, and 7B, for example, a member of a heterogeneous catalyst library of a quaternary composition comprising elements A, B, C and D is advantageously prepared using the afore-described selective active-phase synthesis protocols (FIG. 7B) rather than the conventional, single-pot synthesis protocols described to date in connection with the preparation of combinatorial heterogeneous catalyst libraries (FIG. 7A). Single-pot synthesis can result in a number of detrimental or inactive phases (e.g., BCD, AD, ABC, AC, CD, BC, and BD as shown in FIG. 7A), that can lead to false negatives during combinatorial, high-throughput screening, despite the fact that active or promoter phases (e.g., AB and CD as shown in FIG. 7A) are also present in the candidate catalyst compositions being screened. Selective active-phase synthesis results only in active and promoter phases present in the candidate catalyst compositions (e.g., AB and CD as shown in FIG. 7B).

Advantageously, known or determined active or promoter phases can be preformed and provided as separate source materials—indeed as a source library—useful for subsequent preparation of candidate catalyst libraries. Such a heterogeneous catalyst source library, or “kit”, can comprise, for example, a plurality, and preferably four or more, ten or more, twenty or more, forty or more, seventy or more or one-hundred or more different components, each component comprising one or more active or promoter phases for one or more different reactions of interest or classes of reactions of interest (e.g, dehydrogenation reactions). The source library or “kit” can comprise a plurality (and preferably three or more, four or more or higher numbers) of different catalyst platforms, and can also comprise a plurality of (and preferably three or more, four or more, or higher numbers of) different catalytic functionality—with various capabilities for effecting, promoting, activating or enhancing the making or breaking of a particular type of covalent bond. Exemplary components for which active or promoter phases are known include, for example, acidic functionality for C—H bond activation, oxygen insertion functionality for C oxidation, oxygen activation functionality for oxidation reactions, proton abstraction functionality for dehydrogenations, dehydrocoupling, dehydrodimerization, selective oxidations, aminations, or ammoxidations, and dehydrogenation functionality for dehydrogenation reactions. Further functionalities of interest are defined in the literature—both generally for heterogeneous catalysis, and specifically with respect to particular reactions of interest or classes of reactions. Exemplary components can be based on various catalyst platforms (e.g, polyoxometallates (POM's) with different cations, supports, noble metals, or zeolites, etc.), and can have different functionalities and/or various degrees of the same functionalities. The arrangement and/or organization of the source library or kit is not narrowly critical. Conveniently, however, the components of the source library can be organized (e.g, grouped) and/or selected with respect to particular catalytic functionality and/or by catalyst platform type.

Libraries of catalysts and/or catalyst precursors can be prepared by selecting, and optionally daughtering, the preformed active phases from the source library or kit in various combinations, and physically integrally combining such phases, as described above. Preferably, components comprising two or more different catalytic functionalities are combined to form each of the plurality of candidate catalysts or catalyst precursors of the library. With reference to FIG. 7B, for example, where AB and CD represent, for example, hydrocarbon activation functionality, and oxygen activation functionality, the AB and CD components of a source library or kit can be selected and combined to form the integral AB/CD two-phase composition, useful for effecting reactions involving oxidation of C atoms, with an essential, substantial absence of detrimental phases for the oxidation reaction. The combinatorial library can comprise candidate catalyst or catalyst precursors that vary with respect to each other in terms of amounts (e.g., concentrations) of AB phase relative to CD phase, and/or with respect to other components (e.g, promoters, dopants impregnated or otherwise combined with the AB/CD phases). In one non-limiting example, a combinatorial library can comprise candidate catalyst prepared by daughtering of a common master batch of diverse catalyst precursors corresponding to the predetermined active phase(s), followed by doping each master batch with varied dopants.

Systems exemplifying the desirability of preforming active or promoter phases of heterogeneous catalysts are known, and have been applied with respect to single catalytic systems. Such systems could be incorporated into combinatorial, high-throughput source libraries (kits), library design and screening applications, as described above. Exemplary systems are listed in Table 3A.

TABLE 3A
Exemplary Active Phase Components for Source Library/Screening

Fe—Sb—P—O (ammoxidation catalyst)
FeSbO4 active phase not formed in the presence of P
FeSbO4 must be preformed as a binary component, then mixed with P
MoBiFeCoNiWPO (propylene oxidation catalyst)
powdered iron molybdate preformed, and then mixed as gel with other
components
VMoNbCuWO (acrylic acid catalyst)
CuMoO4 oxygen pump preformed, and then mixed with other
components

Other applications of such approach will be apparent from the art.

Library Design Screening Based on Catalyst Functionality

In another preferred approach for library design and screening protocols, libraries are comprised of materials comprising particular catalytic functionalities. That is, rather than designing and screening libraries by parametric mapping of compositional space, libraries can be designed and screened according to catalytic functionality space—without regard to material composition or structure. Catalytic functionality refers to capabilities of a material for effecting, promoting, activating or enhancing the making or breaking of a particular type of covalent bond. Exemplary components for which active or promoter phases are known include, for example, acidic functionality for C—H bond activation, oxygen insertion functionality for C oxidation, oxygen activation functionality for oxidation reactions, proton abstraction functionality for dehydrogenations, dehydrocoupling, dehydrodimerization, selective oxidations, aminations, or ammoxidations, and dehydrogenation functionality for dehydrogenation reactions. Further functionalities of interest are defined in the literature—both generally for heterogeneous catalysis, and specifically with respect to particular reactions of interest or classes of reactions. Exemplary materials for a catalyst functionality libary can be based on various catalyst platforms (e.g, polyoxometallates (POM's) with different cations, supports, noble metals, or zeolites, etc.), and can have different functionalities and/or various degrees of the same functionalities.

The arrangement and/or organization of the catalytically functional libraries is not narrowly critical. In one exemplary embodiment, a catalytically functional library of materials can comprise a plurality of members having a common catalytic functionality, without regard to compositional structure, and in preferred embodiments, with a diversity of catalytic platforms.

With respect to screening of such functional libraries, the screen can be effected to determine performance with respect to the ultimate reaction of interest, but is preferably effected with respect to an intermediate reaction, and/or with respect to another reaction (i.e., making or breaking of a covalent bond) which results in either a detectable reaction product and/or other means for detecting performance of the library material with respect to the functionality being screened. In preferred approaches, the screening determines performance of and can be employed to optimize functional preformed components for a catalyst candidate for a particular reaction of interest, where such components, after individual optimization by functional screening, will be subsequently combined into a multi-phase catalyst composition (e.g, as described above in connection with the heterogeneous catalysis kit concept).

Various systems are known in the literature as having specific catalytic functionality.

Acidic functionality can be provided, for example, using components such as (Cr, Mo, W)/Al203, zeolites, POM's, H3P04/SiO2, resins with sulfonic acid groups like Amberlyst, Nafion/silica composites, BF3/Al203, TiO2-SiO2, Al203-SiO2 or even homogeneous acids like AlC13, BF3, HF, etc. Known dehydrogenation functionality include components such as noble metals, Mo—V—Nb, Mg-vanadates, K-bismutates, base metal oxides (CuO, NiO, Co3O4), K—(Cr, Fe)/Al203, among others.

A 2-function combinatorial library according to the invention with an acidic and an dehydrogention function would consist of all the permutations of the above subsystems. For example, a library comprising such combined functinoalities could be arranged or configured as, for example: Pt+W/Al203 in the (row 1, col 1) element of the library, Pt+HY zeolite in the (row 1, col 2) element of the library, Mo—V—Nb+W/Al203 in the (row 2, col 1) element of the library, etc. as further shown in Table 2A:

TABLE 2A
Exemplary Catalytic Functionality Library

| | W/ | HY- | HPW- | H3PO4/ | Nafion/ |
|---|---|---|---|---|---|
| | Al2O3 | zeolite | POM | silica | silica |
| Pt | 1, 1 | 1, 2 | 1, 3 | 1, 4 | 1, 5 |
| MoVNb | 2, 1 | 2, 2 | 2, 3 | 2, 4 | 2, 5 |
| Mg3(VO4)2 | 3, 1 | 3, 2 | | | |
| KBiO3 | 4, 1 | 4, 2 | | | |
| Co3O4 | 5, 1 | 5, 2 | | | |
| K—Cr/Al2O3 | 6, 1 | 6, 2 | 6, 3 | 6, 4 | 6, 5 |

The functionality of various components and/or subsystems of complex commercial catalysts for oxidation catalysis is known in the art. (Grasselli, Rimini, 1999), and is summarized in the following Table 2B:

TABLE 2B
Component Functionality of Various Commercial Catalysts

| element/phase | function |
|---|---|
| Acrylonitrile catalyst: | |
| (K, Cs) | modifier |
| (Ni, Mg, Mn, Co) | host |
| (Fe, Ce, Cr) | redox |
| BiMoO | H abstraction, active phase |
| FeMoO | reoxidation |
| (P, Sb) | stabilizer |
| SiO2 | carrier |
| Acrylonitrile catalyst: | |
| Na | modifier |
| (Cu, Mg, Zn, Ni) | modifier/host |
| (V, W) | redox |
| SbO | H abstraction + O insertion, |
| | active phase |
| FeMoO | host, reoxidation of Sb |
| Te | selectivity enhancement |
| SiO2 | carrier |
| SHC(Selective Hydrogen Combustion - | |
| to shift the equilibrium in | |
| dehydrogenations): | |
| PtSn/Al2O3 | oxidative dehydrogenation |
| (CsPt/Al2O3, | hydrogen combustion |
| BiInO, Sb2O4, | |
| BiMoO, InMoO) | |

These completely different catalyst platforms each have the same catalytic functionality—namely H2 combustion to H2O. Hence, an exemplary library according to the present invention, where a H2 combustion functionality is desired to be screened could contain the phases listed in these phases for combinatorialization.

Additionally, catalytic functionality can be characterized with respect to overall conversions, in a multi-step mechanism. As exemplified for propane to acrylic acid conversion, the functionality of the components of the commercial catalyst, VMoNbTeBiO, can be considered as listed in Table 2C.

TABLE 2C
Functionality of Components for Propane to Acrylic Acid Conversion

| | |
|---|---|
| VNb | propane dehydrogenation to propylene |
| BiTeMo | propylene oxidation to acrolein |
| VMo | acrolein oxidation to acrylic acid |

Hence, screening of multi function libraries clarifies the role of each function, to provide a basis for evaluating whether, or to what extent, a particular function is required for the particular reaction of interest (e.g. whether and if weak, medium, strong acidic sites are needed).

Specific Combinations Library and Screening Platforms

Particular library and screening platforms, comprising various combinations of particular elements have been found to be particularly advantageous in a combinatorial (i.e., high-throughput) research program directed to heterogeneous catalysis. The various platforms, together with the constituent groups of elements are summarized below. As screened, the listed elements are, in most cases, present as oxides of the elements formed, in various stable or quasi-stable oxidation states, during calcination and/or other pretreatment steps. Additional components (e.g., dopants) can be included with respect to each of the described platforms. For example, each of the catalyst candidates or catalyst precursors can be doped with one or more alkaline metals, alkaline earth metals, rare earth metals, and/or noble metals. In use, libraries comprising candidate catalysts comprising various combinations and/or concentrations of the components are screened in parallel flow reactors to evaluate the relative catalytic performance, and ultimately to identify new catalysts for a particular reaction of interest.

Redox-Acidic-Dispersant

One preferred library/screening platform comprises a plurality of candidate catalysts, where each of the plurality of different candidate catalysts comprises, or alternatively, consists essentially of, a redox component, an acidic component and a dispersant component. Preferred redox components, acidic components, and dispersants are preferably selected from the groups listed in Table 1A, respectively. The dispersants can be provided, for example, as supports, such as alumina, titania, zirconia, or silica. Such a platform can be applied, for example, to investigate the conversion of alkanes (e.g., ethane) to their corresponding oxygenates.

TABLE 1A
Redox - Acidic - Dispersant Platform

Redox Component: V, Cr, Mo, Co, Ni, Cu, Ag, Bi, Ce, Nd, Sm, Mn
Acidic Component: Ti, Nb, Ta, Mo, W, Fe, B, Al, Si, P
Dispersant Component: Zr, Ti, Al, Si

Redoxl-Redox2-Dispersant

Another library screening platform can comprise a plurality of candidate catalysts, where each of the plurality of different candidate catalysts comprises, or alternatively consists essentially of, a first redox component, a second redox component and a dispersant component. The first redox component is an easily reducible redox component. The second redox component is a difficult to reduce redox component. The dispersant component can also have an acidic functionality, and can be provided, for example, as supports, such as alumina, titania, zirconia, or silica. Preferred first redox components, second redox components, and dispersants are preferably selected from the groups listed in Table 1B, respectively. Such a platform can be applied, for example, to investigate ammoxidations, hydrocarbon dehydrogenation or oxidation to oxygenates.

TABLE 1B
Redox1 - Redox2 - Dispersant Platform

| First Redox | Second Redox | |
|---|---|---|
| Component | Component | Dispersant |
| V | Sb | Si |
| Mo | Sn | Al |
| Cr | In | Ti |
| Mn | Nb | Zr |
| Ni | W | Ta |
| Cu | Pb | P |
| Co | Bi | |
| Ce | Te | |
| Ag | Fe | |
| Ru | | |

Noble Metal-(Noble Metal, Metal Oxide)-Metal Oxide

Another library screening platform can comprise a plurality of candidate catalysts, where each of the plurality of different candidate catalysts comprises, or alternatively consists essentially of either (i) a first noble metal component, a second noble metal component and a metal oxide component, or alternatively, (ii) a noble metal component, a first metal oxide component, and a second metal-oxide component. The metal oxide component can be a transition metal oxide, a rare-earth metal oxide, an alkaline earth metal oxide or other metal oxide. The metal oxide can be provided as a separate component of a catalyst precursor, including, for example, as supports, such as alumina, titania, zirconia, or silica. Preferred noble metals include Pt, Pd, Ru, Rh, Ir, Au and Ag.

Functional Factors

Another library screening platform can comprise a plurality of candidate catalysts, where each of the plurality of different candidate catalysts comprises, or alternatively consists essentially of components identified as being of functional importance for heterogeneous catalysis reactions, in particular, for ammoxidation and hydrocarbon oxidation to oxygenates. The components can be selected from one or more of a redox component, a hydrogen abstraction component, a stabilizer component, an oxygen-insertion component, a host component, and a modifier component, as well as optional dispersants and/or binders, and preferably selected from the groups listed in Table 1C, respectively. Such a platform can be applied, for example, to investigate oxidation and/or ammoxidation catalysis reactions.

TABLE 1C
Functional-Factors Based Platform

| | Hydrogen | | Oxygen | | |
|---|---|---|---|---|---|
| Redox | Abstraction | Stabilizer | Insertion | Host | Modifier |
| Fe | Bi | P | Mo | Co | alkali |
| Ce | Te | As | W | Ni | |
| Cr | In | Sb | V | Mg | |
| Sm | | Nb | Mn | | |
| Tb | | Sn | | | |
| Pr | | Pb | | | |

An alternative library screening platform, based on a modification of the above factors, can comprise a plurality of candidate catalysts, where each of the plurality of different candidate catalysts comprises, or alternatively consists essentially of five components, including a redox component, a hydrogen abstraction component, an oxygen-insertion component, a host component, and a dispersant component, and preferably selected from the groups listed in Table 1D, respectively. Such a platform can be applied, for example, to investigate oxidation and/or ammoxidation catalysis reactions.

TABLE 1D
Functional-Factors Based Platform

| | Hydrogen | Oxygen | | |
|---|---|---|---|---|
| Redox | Abstraction | Insertion | Host | Dispersant |
| Fe | Bi | Mo | Co | Si |
| Ce | Te | V | Ni | Al |
| Cr | In | Mn | Cu | Ti |
| Sb | | W | Mg | Zr |
| Sn | | | | |
| Nb | | | | |
| Pb | | | | |

Other features, objects and advantages of the present invention will be in part apparent to those skilled in art and in part pointed out hereinafter. All references cited in the instant specification are incorporated by reference for all purposes. Moreover, as the patent and non-patent literature relating to the subject matter disclosed and/or claimed herein is substantial, many relevant references are available to a skilled artisan that will provide further instruction with respect to such subject matter.

In light of the detailed description of the invention and the examples presented above, it can be appreciated that the several objects of the invention are achieved.

The explanations and illustrations presented herein are intended to acquaint others skilled in the art with the invention, its principles, and its practical application. Those skilled in the art may adapt and apply the invention in its numerous forms, as may be best suited to the requirements of a particular use. Accordingly, the specific embodiments of the present invention as set forth are not intended as being exhaustive or limiting of the invention.

We claim:

1. A method for evaluating process conditions for a catalyzed chemical reaction in a parallel flow reactor, the method comprising
simultaneously supplying one or more reactants to each of six or more reactors of a parallel flow reactor under reaction conditions to effect a chemical reaction of interest, each of the six or more reactors comprising a catalyst having activity for the chemical reaction, each of the six or more catalysts having substantially the same composition,
controllably varying a set of reaction conditions between each of the six or more reactors, the varied set of reaction conditions comprising (i) at least three different space velocities or contact times or combinations thereof, and (ii) at least two different temperatures, pressures or feed compositions,
simultaneously discharging a reactor effluent from each of the six or more reactors, and
analyzing the reactor effluent from each of the six or more reactors to determine the conversion of one or more reactants and the selectivity for one or more products of the chemical reaction of interest in each of the reactors.

2. The method of claim 1 wherein the set of reaction conditions are varied such that a determined conversion of a conversion-limiting reactant for the six or more reactions includes a range of values that span at least about 10% conversion difference.

3. The method of claim 1 wherein the set of reaction conditions are varied such that a determined conversion of a conversion-limiting reactant for the six or more reactions includes a range of values that span at least about 20% conversion difference.

4. The method of claim 1 wherein the set of reaction conditions are varied such that a determined conversion of a conversion-limiting reactant for the six or more reactions includes a range of values that span at least about 40% conversion difference.

5. The method of claim 1 wherein the set of reaction conditions are varied such that a determined conversion of a conversion-limiting reactant for the six or more reactions includes a range of six values ranging from less than about 20% to more than about 40%.

6. The method of claim 1 wherein the set of reaction conditions are varied such that a determined conversion of a conversion-limiting reactant for the six or more reactions includes a range of six values ranging from less than about 10% to more than about 50%.

7. The method of claim 1 wherein the set of reaction conditions are varied such that a determined conversion of a conversion-limiting reactant for the six or more reactions includes a range of six values ranging from less than about 10% to more than about 70%.

8. The method of claim 1 wherein the set of reaction conditions are varied such that a determined conversion of a conversion-limiting reactant for the six or more reactions includes a range of six values ranging from less than about 2% to more than about 5%.

9. The method of claim 1 wherein the set of reaction conditions are varied such that a determined conversion of a conversion-limiting reactant for the six or more reactions includes a range of six values ranging from less than about 1% to more than about 10%.

10. The method of claim 1 wherein the reactor effluent from each of the six or more reactors are simultaneously analyzed.

11. The method of claim 1 wherein the varied set of reaction conditions comprises (i) at least three different space velocities, and (ii) at least two different temperatures.

12. The method of claim 1 wherein the varied set of reaction conditions comprises (i) at least three different space velocities, and (ii) at least two different pressures.

13. The method of claim 1 wherein the varied set of reaction conditions comprises (i) at least three different space velocities, and (ii) at least two different feed compositions.

14. The method of claim 1 wherein the varied set of reaction conditions comprises (i) at least three different contact times, and (ii) at least two different temperatures.

15. The method of claim 1 wherein the varied set of reaction conditions comprises (i) at least three different contact times, and (ii) at least two different pressures.

16. The method of claim 1 wherein the varied set of reaction conditions comprises (i) at least three different contact times, and (ii) at least two different feed compositions.

17. The method of claim 1 wherein
one or more reactants are simultaneously supplied to each of twelve or more reactors of a parallel flow reactor under reaction conditions to effect a chemical reaction of interest, each of the twelve or more reactors comprising a catalyst having activity for the chemical reaction, each of the twelve or more catalysts having substantially the same composition,
a first set of reaction conditions is controllably varied between each of a first set of six or more reactors, the first set of reaction conditions comprising (i) at least three different space velocities, and (ii) at least two different temperatures, the method further comprising
controllably varying a second set of reaction conditions between each of a second set of six or more reactors, the second set of reaction conditions comprising (i) at least three different space velocities, and (ii) at least two different feed compositions.

18. The method of claims 1 or 17 wherein each of the catalysts are prepared under substantially the same conditions.

19. The method of claims 1 or 17 wherein each of the catalysts are substantially the same catalysts.

20. A method for evaluating process conditions for a catalyzed chemical reaction in a parallel flow reactor, the method comprising
simultaneously supplying one or more reactants to each of six or more reactors of a parallel flow reactor under reaction conditions to effect a chemical reaction of interest, each of the six or more reactors comprising a catalyst having activity for the chemical reaction, each of the six or more catalysts having substantially the same composition,
controllably varying a set of reaction conditions between each of the six or more reactors, the varied set of reaction conditions comprising (i) at least three different space velocities, and (ii) at least two different contact times,
simultaneously discharging a reactor effluent from each of the six or more reactors, and
analyzing the reactor effluent from each of the six or more reactors to determine the conversion of one or more reactants and the selectivity for one or more products of the chemical reaction of interest in each of the reactors.

21. The method of claim 20 wherein the set of reaction conditions are varied such that a determined conversion of a conversion-limiting reactant for the six or more reactions includes a range of values that span at least about 10% conversion difference.

22. The method of claim 20 wherein the set of reaction conditions are varied such that a determined conversion of a conversion-limiting reactant for the six or more reactions includes a range of values that span at least about 20% conversion difference.

23. The method of claim 20 wherein the set of reaction conditions are varied such that a determined conversion of a conversion-limiting reactant for the six or more reactions includes a range of six values ranging from less than about 2% to more than about 5%.

24. The method of claim 20 wherein the set of reaction conditions are varied such that a determined conversion of a conversion-limiting reactant for the six or more reactions includes a range of six values ranging from less than about 1% to more than about 10%.

25. The method of claim 20 wherein the set of reaction conditions are varied such that a determined conversion of the conversion-limiting reactant for the six or more reactions includes a range of six values ranging from less than about 20% to more than about 40%.

26. The method of claim 20 wherein the range of six values for the determined conversion ranges from about 10% to about 50%.

27. A method for evaluating process conditions for a catalyzed chemical reaction in a parallel flow reactor, the method comprising
simultaneously supplying one or more reactants to each of twelve or more reactors of a parallel flow reactor under reaction conditions to effect a chemical reaction of interest, each of the twelve or more reactors comprising a catalyst having activity for the chemical reaction, a first set of six or more of the catalysts having substantially the same first composition, a second set of six or more of the catalysts having substantially the same second composition, the second composition being different from the first composition,
controllably varying a first set of reaction conditions between each of the six or more reactors comprising the first set of catalysts, the varied first set of reaction conditions comprising (i) at least three different space velocities or contact times or combinations thereof, and (ii) at least two different temperatures, pressures or feed compositions,
controllably varying a second set of reaction conditions between each of the six or more reactors comprising the second set of catalysts, the varied second set of reaction conditions comprising (i) at least three different space velocities or contact times or combinations thereof, and (ii) at least two different temperatures, pressures or feed compositions,
simultaneously discharging a reactor effluent from each of the twelve or more reactors, and
analyzing the reactor effluent from each of the twelve or more reactors to determine the conversion of one or more reactants and the selectivity for one or more products of the chemical reaction of interest in each of the reactors.

28. The method of claim 27 wherein the first set of reaction conditions are varied such that a determined conversion of a conversion-limiting reactant for the six or more reactions catalyzed by the first set of catalysts includes a range of values that span at least about 10% conversion difference, and the second set of reaction conditions are varied such that the determined conversion of the conversion-limiting reactant for the six or more reactions catalyzed by the second set of catalysts includes a range of values that span at least about 10% conversion difference.

29. The method of claim 27 wherein the first set of reaction conditions are varied such that determined conversion of a conversion-limiting reactant for the six or more reactions catalyzed by the first set of catalysts includes a range of values that span at least about 20% conversion difference, and the second set of reaction conditions are varied such that the determined conversion of the conversion-limiting reactant for the six or more reactions catalyzed by the second set of catalysts includes a range of values that span at least about 20% conversion difference.

30. The method of claim 27 wherein the first set of reaction conditions are varied such that a determined conversion of a conversion-limiting reactant for the six or more reactions catalyzed by the first set of catalysts includes a range of values that span at least about 40% conversion difference, and the second set of reaction conditions are varied such that the determined conversion of the conversion-limiting reactant for the six or more reactions catalyzed by the second set of catalysts includes a range of values that span at least about 40% conversion difference.

31. The method of claim 27 wherein the first set of reaction conditions are varied such that a determined conversion of a conversion-limiting reactant for the six or more reactions catalyzed by the first set of catalysts includes a range of six values ranging from less than about 20% to more than about 40%, and the second set of reaction conditions are varied such that the determined conversion of the conversion-limiting reactant for the six or more reactions catalyzed by the second set of catalysts includes a range of six values ranging from less than about 20% to more than about 40%.

32. The method of claim 27 wherein the first set of reaction conditions are varied such that a determined conversion of a conversion-limiting reactant for the six or more reactions catalyzed by the first set of catalysts includes a range of six values ranging from less than about 10% to more than about 50%, and the second set of reaction conditions are varied such that the determined conversion of the conversion-limiting reactant for the six or more reactions catalyzed by the second set of catalysts includes a range of six values ranging from less than about 10% to more than about 50%.

33. The method of claim 27 wherein the first set of reaction conditions are varied such that a determined conversion of a conversion-limiting reactant for the six or more reactions catalyzed by the first set of catalysts includes a range of six values ranging from less than about 10% to more than about 70%, and the second set of reaction conditions are varied such that the determined conversion of the conversion-limiting reactant for the six or more reactions catalyzed by the second set of catalysts includes a range of six values ranging from less than about 10% to more than about 70%.

34. The method of claim 27 wherein the first set of reaction conditions are varied such that a determined conversion of a conversion-limiting reactant for the six or more reactions catalyzed by the first set of catalysts includes a range of six values ranging from less than about 2% to more than about 5%, and the second set of reaction conditions are varied such that the determined conversion of the conversion-limiting reactant for the six or more reactions catalyzed by the second set of catalysts includes a range of six values ranging from less than about 2% to more than about 5%.

35. The method of claim 27 wherein the first set of reaction conditions are varied such that a determined conversion of a conversion-limiting reactant for the six or more reactions catalyzed by the first set of catalysts includes a range of six values ranging from less than about 1% to more than about 10%, and the second set of reaction conditions are varied such that the determined conversion of the conversion-limiting reactant for the six or more reactions catalyzed by the second set of catalysts includes a range of six values ranging from less than about 1% to more than about 10%.

36. The method of claim 27 wherein the reactor effluent from each of the twelve or more reactors are simultaneously analyzed.

37. The method of claim 27 wherein the varied first and second sets of reaction conditions each comprise (i) at least three different space velocities, and (ii) at least two different temperatures.

38. The method of claim 27 wherein the varied first and second sets of reaction conditions each comprise (i) at least three different space velocities, and (ii) at least two different pressures.

39. The method of claim 27 wherein the varied first and second sets of reaction conditions each comprise (i) at least three different space velocities, and (ii) at least two different feed compositions.

40. The method of claim 27 wherein the varied first and second sets of reaction conditions each comprise (i) at least three different contact times, and (ii) at least two different temperatures.

41. The method of claim 27 wherein the varied first and second sets of reaction conditions each comprise (i) at least three different contact times, and (ii) at least two different pressures.

42. The method of claim 27 wherein the varied first and second sets of reaction conditions each comprise (i) at least three different contact times, and (ii) at least two different feed compositions.

43. The method of claim 27 wherein each of the six or more catalysts of the first set of catalysts are prepared under substantially the same first set of preparation conditions, and each of the six or more catalysts of the second set of catalysts are prepared under substantially the same second set of preparation conditions, the first set and second set of preparation conditions being the same as or different from each other.

44. The method of claim 27 wherein each of the six or more catalysts of the first set of catalysts are substantially the same first catalysts, and each of the six or more catalysts of the second set of catalysts are substantially the same second catalysts.

45. A method for evaluating process conditions for a catalyzed chemical reaction in a parallel flow reactor, the method comprising
simultaneously supplying one or more reactants to each of twelve or more reactors of a parallel flow reactor under reaction conditions to effect a chemical reaction of interest, each of the twelve or more reactors comprising a catalyst having activity for the chemical reaction, a first set of six or more of the catalysts having substantially the same first composition, a second set of six or more of the catalysts having substantially the same second composition, the second composition being different from the first composition,
controllably varying a first set of reaction conditions between each of the six or more reactors comprising the first set of catalysts, the varied first set of reaction conditions comprising (i) at least three different space velocities, and (ii) at least two different contact times,
controllably varying a second set of reaction conditions between each of the six or more reactors comprising the second set of catalysts, the varied second set of reaction conditions comprising (i) at least three different space velocities, and (ii) at least two different contact times,
simultaneously discharging a reactor effluent from each of the twelve or more reactors, and
analyzing the reactor effluent from each of the twelve or more reactors to determine the conversion of one or more reactants and the selectivity for one or more products of the chemical reaction of interest in each of the reactors.

46. The method of claim 45 wherein the first set of reaction conditions are varied such that a determined conversion of a conversion-limiting reactant for the six or more reactions catalyzed by the first set of catalysts includes a range of six values that span at least about 10% conversion difference, and the second set of reaction conditions being varied such that the determined conversion of the conversion-limiting reactant for the six or more reactions catalyzed by the second set of catalysts includes a range of six values that span at least about 10% conversion difference.

47. The method of claim 45 wherein the first set of reaction conditions are varied such that a determined conversion of a conversion-limiting reactant for the six or more reactions catalyzed by the first set of catalysts includes a range of six values that span at least about 20% conversion difference, and the second set of reaction conditions being varied such that the determined conversion of the conversion-limiting reactant for the six or more reactions catalyzed by the second set of catalysts includes a range of six values that span at least about 20% conversion difference.

48. The method of claim 45 wherein the first set of reaction conditions are varied such that a determined conversion of a conversion-limiting reactant for the six or more reactions catalyzed by the first set of catalysts includes a range of six values ranging from less than about 20% to more than about 40%, and the second set of reaction conditions are varied such that the determined conversion of the conversion-limiting reactant for the six or more reactions catalyzed by the second set of catalysts includes a range of six values ranging from less than about 20% to more than about 40%.

49. The method of claim 45 wherein the first set of reaction conditions are varied such that a determined conversion of a conversion-limiting reactant for the six or more reactions catalyzed by the first set of catalysts includes a range of six values ranging from less than about 10% to more than about 50%, and the second set of reaction conditions being varied such that the determined conversion of the conversion-limiting reactant for the six or more reactions catalyzed by the second set of catalysts includes a range of six values ranging from less than about 10% to more than about 50%.

50. The method of claim 45 wherein the first set of reaction conditions are varied such that a determined conversion of a conversion-limiting reactant for the six or more reactions catalyzed by the first set of catalysts includes a range of six values ranging from less than about 2% to more than about 5%, and the second set of reaction conditions being varied such that the determined conversion of the conversion-limiting reactant for the six or more reactions catalyzed by the second set of catalysts includes a range of six values ranging from less than about 2% to more than about 5%.

51. The method of claim 45 wherein the first set of reaction conditions are varied such that a determined conversion of a conversion-limiting reactant for the six or more reactions catalyzed by the first set of catalysts includes a range of six values ranging from less than about 1% to more than about 10%, and the second set of reaction conditions being varied such that the determined conversion of the conversion-limiting reactant for the six or more reactions catalyzed by the second set of catalysts includes a range of six values ranging from less than about 1% to more than about 10%.

52. A method for evaluating process conditions for a catalyzed chemical reaction in a parallel flow reactor, the method comprising
simultaneously supplying one or more reactants to each of sixteen or more reactors of a parallel flow reactor under reaction conditions to effect a chemical reaction of interest, each of the sixteen or more reactors comprising a catalyst having activity for the chemical reaction, the sixteen or more reactors comprising a first set of four or more of the catalysts having substantially the same first composition, a second set of four or more of the catalysts having substantially the same second composition, a third set of four or more of the catalysts having substantially the same third composition, and a fourth set of four or more of the catalysts having substantially the same fourth composition, each of the first composition, the second composition, the third composition and the fourth composition being different from each other,
controllably varying a set of reaction conditions between each of the sixteen or more reactors, the varied set of reaction conditions comprising (i) at least four different space velocities or contact times or combinations thereof, and (ii) at least four different temperatures, pressures or feed compositions, the set of reaction conditions being varied such that at least one catalyst from each of the first, second, third and fourth sets of catalysts catalyzes the chemical reaction under each of the at least four different space velocities or contact times or combinations thereof, and under each of the at least four different temperatures, pressures or feed compositions,
simultaneously discharging a reactor effluent from each of the sixteen or more reactors, and
analyzing the reactor effluent from each of the sixteen or more reactors to determine the conversion of one or more reactants and the selectivity for one or more products of the chemical reaction of interest in each of the reactors.

53. The method of claim 52 wherein the set of reaction conditions is varied such that a determined conversion of a conversion-limiting reactant for each of (a) the four or more reactions catalyzed by the first set of catalysts, (b) the four or more reactions catalyzed by the second set of catalysts, (c) the four or more reactions catalyzed by the third set of catalysts, and (d) the four or more reactions catalyzed by the forth set of catalysts, includes a range of six values that span at least about 10% conversion difference.

54. The method of claim 52 wherein the set of reaction conditions is varied such that a determined conversion of a conversion-limiting reactant for each of (a) the four or more reactions catalyzed by the first set of catalysts, (b) the four or more reactions catalyzed by the second set of catalysts, (c) the four or more reactions catalyzed by the third set of catalysts, and (d) the four or more reactions catalyzed by the forth set of catalysts, includes a range of six values that span at least about 20% conversion difference.

55. The method of claim 52 wherein the set of reaction conditions is varied such that a determined conversion of a conversion-limiting reactant for each of (a) the four or more reactions catalyzed by the first set of catalysts, (b) the four or more reactions catalyzed by the second set of catalysts, (c) the four or more reactions catalyzed by the third set of catalysts, and (d) the four or more reactions catalyzed by the forth set of catalysts, includes a range of six values that span at least about 40% conversion difference.

56. The method of claim 52 wherein the set of reaction conditions is varied such that a determined conversion of a conversion-limiting reactant for each of (a) the four or more reactions catalyzed by the first set of catalysts, (b) the four or more reactions catalyzed by the second set of catalysts, (c) the four or more reactions catalyzed by the third set of catalysts, and (d) the four or more reactions catalyzed by the forth set of catalysts, includes a range of six values ranging from less than about 20% to more than about 40%.

57. The method of claim 56 wherein range of six values ranges from less than about 10% to more than about 50%.

58. The method of claim 56 wherein range of six values ranges from less than about 10% to more than about 70%.

59. The method of claim 52 wherein the set of reaction conditions is varied such that a determined conversion of a conversion-limiting reactant for each of (a) the four or more reactions catalyzed by the first set of catalysts, (b) the four or more reactions catalyzed by the second set of catalysts, (c) the four or more reactions catalyzed by the third set of catalysts, and (d) the four or more reactions catalyzed by the forth set of catalysts, includes a range of six values ranging from less than about 2% to more than about 5%.

60. The method of claim 52 wherein the set of reaction conditions is varied such that a determined conversion of a conversion-limiting reactant for each of (a) the four or more reactions catalyzed by the first set of catalysts, (b) the four or more reactions catalyzed by the second set of catalysts, (c) the four or more reactions catalyzed by the third set of catalysts, and (d) the four or more reactions catalyzed by the forth set of catalysts, includes a range of six values ranging from less than about 1% to more than about 10%.

61. The method of claim 52 wherein one or more reactants are simultaneously supplied to each of twenty or more reactors of a parallel flow reactor to effect the chemical reaction of interest, each of the twenty or more reactors comprises a catalyst having activity for the chemical reaction, and four or more of the reactors comprise a reference set of four or more reference catalysts having substantially the same reference composition, each of the four or more reference catalysts catalyzing the chemical reaction under reaction conditions that correspond to four or more of the controllably varied reaction conditions.

62. The method of claim 61 further comprising, for at least one of the first, second, third or fourth catalyst compositions, interpolating or extrapolating at least a portion of a master curve for the reaction of interest defined by a plot of the determined selectivity versus the determined conversion of the reaction of interest for the at least one catalyst composition.

63. The method of claim 62 wherein at least a portion of the master curve is interpolated or extrapolated by comparison with a master curve for the eight or more reference catalysts.

64. The method of claim 52 wherein one or more reactants are simultaneously supplied to each of twenty or more reactors of a parallel flow reactor to effect the chemical reaction of interest, at least sixteen or more reactors comprises a catalyst having activity for the chemical reaction, and at least two or more of the reactors are blank reactors having an essential absence of catalysts having activity for the reaction of interest.

65. The method of claim 52 wherein the reactor effluent from each of the twelve or more reactors are simultaneously analyzed.

66. The method of claim 52 wherein the varied set of reaction conditions comprises (i) at least three different space velocities, and (ii) at least two different temperatures.

67. The method of claim 52 wherein the varied set of reaction conditions comprises (i) at least three different space velocities, and (ii) at least two different pressures.

68. The method of claim 52 wherein the varied set of reaction conditions comprises (i) at least three different space velocities, and (ii) at least two different feed compositions.

69. The method of claim 52 wherein the varied set of reaction conditions comprises (i) at least three different contact times, and (ii) at least two different temperatures.

70. The method of claim 52 wherein the varied set of reaction conditions comprises (i) at least three different contact times, and (ii) at least two different pressures.

71. The method of claim 52 wherein the varied set of reaction conditions comprises (i) at least three different contact times, and (ii) at least two different feed compositions.

72. The method of claim 52 wherein one or more reactants are simultaneously supplied to each of twenty-four or more reactors of a parallel flow reactor to effect the chemical reaction of interest, each of the twenty-four or more reactors comprises a catalyst having activity for the chemical reaction, and the varied set of reaction conditions comprise (i) at least six different space velocities or contact times or combinations thereof, and (ii) at least four different temperatures, pressures or feed compositions, the set of reaction conditions being varied such that at least one catalyst from each of the first, second, third and fourth sets of catalysts catalyzes the chemical reaction under each of the at least six different space velocities or contact times or combinations thereof, and under each of the at least four different temperatures, pressures or feed compositions.
